(19) United States
(12) Patent Application Publication
(10) Pub. No.: US 2021/0358637 A1
(43) Pub. Date: Nov. 18, 2021

(54) SYSTEM AND METHOD FOR DETECTING ADVERSE MEDICATION INTERACTIONS VIA A WEARABLE DEVICE

(71) Applicants: KONINKLIJKE PHILIPS N.V., EINDHOVEN (NL); PHILIPS NORTH AMERICA LLC, ANDOVER, MA (US)

(72) Inventor: Vikram DEVDAS, Vancouver (CA)

(21) Appl. No.: 17/290,406

(22) PCT Filed: Oct. 31, 2019
(86) PCT No.: PCT/EP2019/079806

§ 371 Date: Apr. 30, 2021

Related U.S. Application Data
(60) Provisional application No. 62/753,844, filed on Oct. 31, 2018.

(51) Int. Cl. G16H 50/70; G16H 50/20; G16H 50/30; G16H 20/10; G16H 10/60; G16H 40/67; G06N 20/00; A61B 5/00; A61B 5/11

(52) U.S. Cl. CPC G16H 50/70; G16H 50/20; G16H 50/30; G16H 20/10; G16H 10/60; G16H 40/67; A61B 5/4848; A61B 5/7275; A61B 5/7267; A61B 5/7282; A61B 5/1117; A61B 5/6801; A61B 5/1113; G06N 20/00

(57) ABSTRACT

The present patent application relates detecting adverse medication interactions using a wearable device. In some embodiments, fall event data of a patient is obtained, the fall event data including a set of fall events experienced by the patient during a first time period. Medication data associated with the patient is also capable of being obtained. The medication data indicates a set of medications taken or prescribed to be taken by the patient during the first time period. Training data is configured to be generated for a prediction model based on the fall event data and the medication data, and the training data is capable of being provided to the prediction model. The prediction model is configured to estimate, based on the training data, a dependency between one or more medications and a risk of experiencing a fall event.

### CROSS-REFERENCE TO RELATED APPLICATION

The present patent application claims the benefit of U.S. Provisional Patent Application No. 62/753,844, entitled “METHOD FOR ESTIMATING FALL RISK DUE TO MEDICATIONS VIA A WEARABLE DEVICE,” which was filed on Oct. 31, 2018, and the disclosure of which is incorporated herein by reference in its entirety.

### BACKGROUND

### Field

The present patent application discloses various systems and methods relating to determining medical interactions.

### Description of the Related Art

Wearable devices are known and used to track movements of individuals. It is also known that certain medications, when prescribed to individuals, can cause adverse effects. For example, an individual may be at a greater risk of falling due to certain medications. As another example, an individual's mobility may increase or decrease due to certain medications. However, current systems have limited ability to use the information obtained via such wearable devices in determining various interactions, such as whether an individual has an increased risk of experiencing adverse effects (such as falling). Furthermore, current systems have limited ability to determine dependencies between medications and an increased risk in an individual experiencing such adverse effects. The present patent application offers improvements in such systems.

### SUMMARY

Accordingly, one or more aspects of the present patent applications relate to a method for training of a prediction model to estimate a fall risk due to medications, the method being implemented by one or more processors executing one or more computer program instructions such that, when executed, the one or more processors effectuate the method, the method comprising: obtaining fall event data of a patient, wherein the fall event data comprises a set of fall events experienced by the patient during a first time period; obtaining medication data associated with the patient, wherein the medication data indicates a set of medications to be taken by the patient during the first time period; generating training data for a prediction model based on the fall event data and the medication data; and providing the training data to the prediction model, the prediction model being configured, based on the training data, to estimate a dependency between one or more of the medications and an increase in a risk of experiencing a fall event.

Another aspect of the present patent application relates to a method for facilitating training of a prediction model to estimate a change in mobility due to medications, the method being implemented by one or more processors executing one or more computer program instructions such that, when executed, the one or more processors effectuate the method, the method comprising: obtaining mobility data of a patient, wherein the mobility data indicates movement of the patient during a first time period; obtaining medication data associated with the patient, wherein the medication data indicates a set of medications taken or prescribed to be taken by the patient during the first time period; generating training data for a prediction model based on the mobility data and the medication data; and providing the training data to the prediction model, the prediction model being configured, based on the training data, to estimate a dependency between one or more of the medications and a change in mobility.

Yet another aspect of the present patent application relates to a system, comprising: a wearable device comprising one or more motion sensors, wherein the patient client device is to be worn by a patient; and a computer system comprising one or more processors operatively coupled to the wearable device and configured to execute computer program instructions such that, when executed, the one or more processors are configured to: monitor a mobility of a patient during a first time period when the patient has not been prescribed any medications; monitor the mobility of the patient during a second time period when the patient has been prescribed one or more medications; and determine whether the patient took the one or more medications based upon a change in the mobility of the patient during the first time as compared to the mobility of the patient during the second time period.

Still yet another aspect of the present patent application relates to a method for generating a warning notification for potential adverse medication effects, the method being implemented by one or more processors executing one or more computer program instructions such that, when executed, the one or more processors effectuate the method, the method comprising: obtaining medication data indicating a set of medications taken or prescribed to be taken by a first patient; providing the medication data to a trained prediction model; and generating a warning notification in response to an output from the trained prediction model indicating that a risk of the set of medications causing the patient adverse medication effects satisfies a threshold risk condition.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows an exemplary system for detecting adverse medication interactions from a wearable device, in accordance with various embodiments;

FIG. 2 shows a schematic illustration of a care facility housing patients, in accordance with various embodiments;

FIG. 3 shows a schematic illustration of a patient's movements within a portion of a care facility and a fall event of the patient, in accordance with various embodiments;

FIG. 4 shows a schematic illustration of mobility data of a patient, in accordance with various embodiments;

FIGS. 5A and 5B show an example medication database and fall event database, respectively, in accordance with various embodiments;

FIG. 6 shows an example training data database including labeled mobility data, in accordance with various embodiments;

FIG. 7 shows another example training data database including labeled fall event data, in accordance with various embodiments;

FIG. 8 shows an example system including an example predication model, in accordance with various embodiments;

FIG. 9 shows an example results database including determined dependencies with respect to medications, in accordance with various embodiments;

FIG. 10 shows an example provider client device displaying a warning notification for a provider, in accordance with various embodiments;

FIG. 11 shows an example method for facilitating training of a prediction model, in accordance with various embodiments;

FIG. 12 shows an example method for determining whether to a patient took one or more prescribed medications, in accordance with various embodiments;

FIG. 13 shows another example method for facilitating training of a prediction model, in accordance with various embodiments;

FIG. 14 shows an example method for determining whether a patient took one or more prescribed medications, in accordance with various embodiments; and

FIG. 15 shows an example computer system for implementing one or more of the aforementioned aspects, in accordance with various embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As employed herein, the term “number” shall mean one or an integer greater than one (i.e., a plurality).

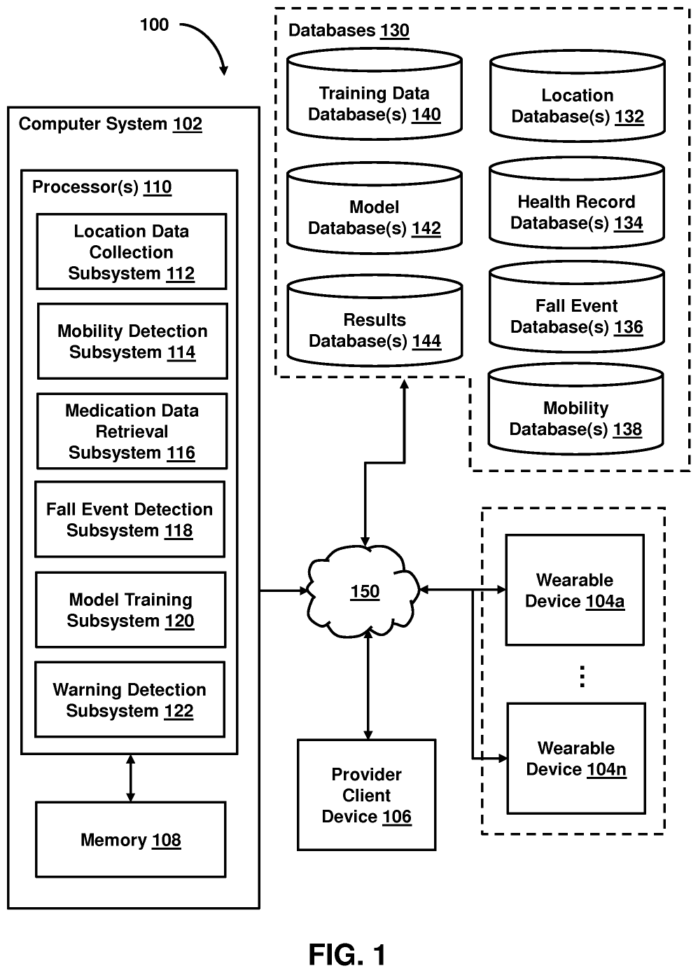

FIG. 1 shows an exemplary system 100 for detecting adverse medication interactions from a wearable device, in accordance with various embodiments. In some embodiments, system 100 a computer system 102 (e.g., one or more servers or other computer systems), wearable devices 104a-104n (collectively referred to as “wearable devices 104”), a provider client device 106, and databases 130. As described herein, each of wearable devices 104a-104n is also referred to interchangeably as wearable device 104. Although only a single provider client device 106 is illustrated, system 100 may include multiple provider client devices that are the same or similar to provider client device 106. Computer system 102, wearable device(s) 104, provider client device 106, and databases 130 are configured to be operatively coupled to one another such that each of computer system 102, wearable device(s) 104, provider client device 106, and databases 130 can communicate with one another, or with other components, devices, and systems, via network 150. For example, network 150 is capable of being accessed by any component of system 100 using Transfer Control Protocol and Internet Protocol (“TCP/IP”) (e.g., any of the protocols used in each of the TCP/IP layers), Hypertext Transfer Protocol (“HTTP”), WebRTC, SIP, and wireless application protocol (“WAP”). In one embodiment, network 150 facilitates communications between components of system 100 or other components with one another via a web browser using HTTP. Various additional communication protocols used to facilitate communications between components of system 100 include, but are not limited to, Wi-Fi (e.g., 802.11 protocol), Bluetooth, radio frequency systems (e.g., 900 MHz, 1.4 GHz, and 5.6 GHz communication systems), cellular networks (e.g., GSM, AMPS, GPRS, CDMA, EV-DO, EDGE, 3GSM, DECT, IS-136/TDMA, iDen, LTE or any other suitable cellular network protocol), infrared, BitTorrent, FTP, RTP, RTSP, SSH, and/or VOIP.

Databases 130 include one or more location databases 132, one or more health record databases 134, one or more fall event databases 136, one or more mobility databases 138, one or more training data databases 140, one or more model databases 142, and one or more results databases 144. In some embodiments, system 100 includes multiple instances of one or more of location database(s) 132, health record database(s) 134, fall event database(s) 136, mobility database(s) 138, training data database(s) 140, model database(s) 142, and results database(s) 144. However, for simplicity, a single instance of each of location database(s) 132, health record database(s) 134, fall event database(s) 136, mobility database(s) 138, training data database(s) 140, model database(s) 142, and results database(s) 144 is described. For example, as described herein, “location database 132” refers to a single location database or multiple location databases. Each of the databases included within databases 130 are capable of being distributed databases, cloud-based storage databases, and the like.

In some embodiments, wearable device 104 is a wrist-worn (watch style) or any other style of wearable device, and can communicate with computer system 102. For example, wearable device 104 communicates periodically, via a wireless or wired connection (e.g., network 150), with computer system 102 and/or databases 130 in order to upload/store recorded location data and fall events. In some embodiments, wearable device 104 is configured to communicate with one or more wireless beacons and/or other wearable devices (that are the same or similar to wearable device 104). By communicating with the wireless beacons, a location of wearable device 104 within a care facility may be determined. For example, the wireless beacons may implement ultra-wideband (UWB) technology or another wireless technology, such as Bluetooth or WiFi, to repeatably locate wearable devices 104 and/or other devices within a given area. In some embodiments, a patient of a care facility is assigned multiple wearable devices 104. For example, an administrator may assign two wearable devices 104 to a patient, including: a first wearable device 104a to be worn by the patient during the day and recharged at night; and a second wearable device 104n to be worn by the patient at night and recharged during the day. Each wearable device 104 can be loaded with a unique ID (e.g., a wireless ID), and the unique ID can be associated with a particular patient of the care facility, such as in a name mapping server (or “NMS”). In some embodiments, as described below, the wearable device can include: a set of inertial sensors; one or more processors configured to classify its motion (e.g., sleeping, sitting, walking, running, and a rate of each) based on outputs of the inertial sensor(s); a geospatial location sensor (e.g., a GPS sensor or an UWB compatible transmitter); a wireless communication module that broadcasts location data; a rechargeable battery that powers the foregoing elements, and/or other components.

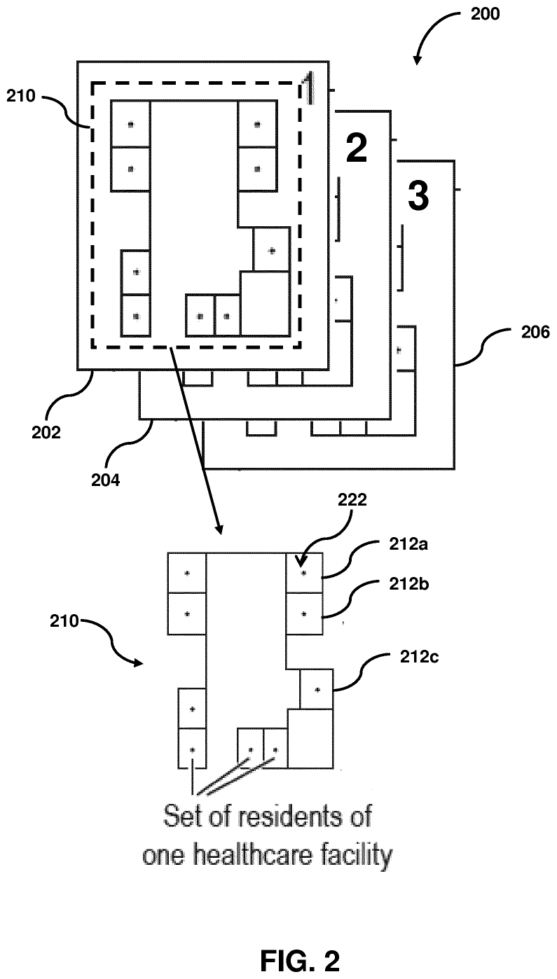

As described herein, a care facility includes general hospitals, psychiatric hospitals, or any other health institution, clinic, or community assisted living communities, hospitals (including psychiatric hospitals), and/or any other type of long-term (e.g., overnight) care facility. The patient adorning wearable device 104 (or multiple wearable devices) can be a temporary patient at a care facility or can be a long-term resident of the care facility. As an example, with reference to FIG. 2, a care facility 200 includes levels or floors 202-206. On each level or floor 202-206, a floorplan 210 is depicted indicating a layout of the various structural aspects of the given floor. For example, floorplan 210 may indicate a shape, orientation, and/or location of a plurality of patient rooms 212a-212c existing on floor 202 of care facility 200. Furthermore, floorplan 210 includes an indicator, identifier, or other identification mechanism of a patient 222 or patients residing in each room. For example, a patient identifier of patient 222 may indicate the patient's name, medical information, room number, floor number, and/or other information. As another example, the patient identifier of patient 222 may be a wearable device identifier of a wearable device (e.g., wearable device 104) worn by patient 222. As described herein, patient 222 may also be referred to interchangeably as “a resident,” such as a resident of care facility 200.

In some embodiments, provider client device 106 is associated with a provider located at care facility 200. For instance, the provider may be a doctor, nurse, technician, emergency medical provider, social worker, family member. Provider client device 106 may be a wearable device (the same or similar to wearable client device 104) and/or one or more mobile computer devices. Provider client device 106 and wearable devices 104 can additionally or alternatively include: a short-range wireless communication module (e.g., a low power 2.4 GHz wireless communication device); an inertial sensor (e.g., an accelerometer and/or gyroscope sensor); an input field (e.g., a touchscreen); a processor; and/or a rechargeable battery. In some embodiments, provider client device 106 includes a computing device (e.g., a desktop or laptop computer, a tablet or a smartphone) assigned to a provider, which can execute a care provider application. For example, the care provider application can: receive a notification from computer system 102 indicating the effects of a combination of prescription medications. Furthermore, the care provider application can interface directly (e.g., as an add-on) to an EHR application in order to provide notifications regarding prescription medications while a care provider is reviewing EHR records for a patient. Alternatively, the care provider application can interface with health records database 134, which may be used by the EHR application, in order to access medication data from the EHR application.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

In some embodiments, computer system 102 includes a location data collection subsystem 112, a mobility detection subsystem 114, a medication data retrieval subsystem 116, a fall event detection subsystem 118, a model training subsystem 120, and a warning detection subsystem 122. Furthermore, computer system 102 may include one or more processors 110, memory 108, and/or other components. Memory 108 may include computer program instructions that, when executed by processors 110, effectuate operations to be performed, including causing the functions of any of subsystems 112-122 to be performed. The computer program instructions may refer to machine-readable instructions stored within memory 108 and executable by processors 110 automatically, in response to a request to perform a particular function or functions, or both.

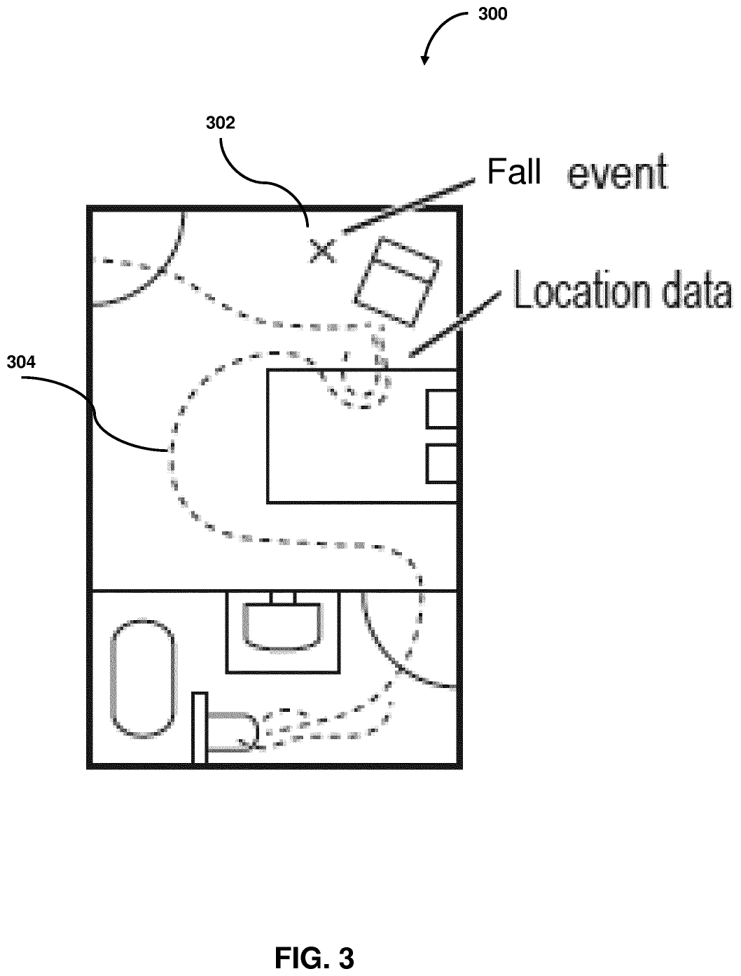

In some embodiments, location data collection subsystem 112 is configured to collect location data from wearable devices 104, each of which is associated with one or more patients of a care facility (e.g., care facility 200). For instance, computer system 102 collects a time series of location data for a patient wearing wearable device 104. As an example, with reference to FIG. 3, a wearable device 104 records movements 304 within a floorplan 300 of a room (e.g., one of rooms 212a-212c) associated with a patient (e.g., patient 222) occupying a care facility (e.g., care facility 200). In some embodiments, computer system 102 interfaces with a set of wireless beacons (e.g., mounted to walls of a facility) to detect a location of wearable device 104 worn by a patient in a particular care facility. Additionally, computer system 102 can track a location of each provider client device 106, based on floorplan 300, which may be a wearable device and/or a computing device, deployed throughout the care facility. For example, computer system 102 may track an absolute geospatial location of the patient within the care facility or a location of the patient relative to one or more wireless beacons or other wireless-enabled devices of known location within the care facility based on floorplan 300. In some embodiments, computer system 102 tracks the location of the patient by leveraging UWB technology. Using UWB technology, a greater degree of accuracy (e.g., to within one meter) of a location of a patient is possible. With greater location resolution, computer system 102 may achieve improved estimation of the patient's activities of daily living (ADLs) for a patient based on the location data.

As described in commonly-assigned U.S. patent application Ser. No. 15/339,771, entitled “METHODS FOR DETECTING AND HANDLING FALL AND PERIMETER BREACH EVENTS FOR RESIDENTS OF AN ASSISTED LIVING FACILITY,” filed on Oct. 31, 2016, and which issued as U.S. Pat. No. 9,922,524 on Mar. 20, 2019, the disclosures of which are incorporated by reference herein in their entireties, to calculate the location of wearable device 104, wearable device 104 can be configured to broadcast a test signal to a set of wireless beacons of known locations within the care facility. Wearable device 104 may receive return signals and wireless IDs from the wireless beacons, calculate a flight time for the test signal, and transmit location data including these wireless IDs and corresponding flight times of the test signals (via a wireless beacon) to computer system 102. For example, location data collection subsystem 112 may receive the location data including the wireless IDs and corresponding flight times of the test signals. In some embodiments, location data collection subsystem 112 is configured to reconstruct the location of wearable device 104—and therefore the patient associated with wearable device 104—from the location data.

In some embodiments, one or more computer program instructions stored within memory of wearable device 104, when executed by one or more processors of wearable device 104, effectuate operations including collecting wireless IDs and test signal flight times from two or more local wireless beacons, and transmitting the wireless IDs and test signal flight times to computer system 102 via a local wireless beacon. Location data collection subsystem 112 of computer system 102 is configured to implement similar techniques to determine the location of the patient within the care facility, such as by locating (e.g., via multilateration) the position of wearable device 104 within the care facility relative to the (e.g., three or more) wireless beacons. Location data collection subsystem 112 of computer system 102 may also be configured to locate wearable device 104 associated with the patient based on proximity to other devices within the care facility. For example, a location of wearable device 104 may be determined based on flight times of test signals broadcast by wearable device 104 and returned from other wearable devices 104 associated with different patients/residents of the care facility, and/or provider client devices 106 within the care facility.

In some embodiments, location data collection subsystem 112 of computer system 102 is configured to determine the location (e.g., a point, an area) of wearable device 104 based on time of flight data received from the set of wireless beacons and/or other wireless-enabled devices in communication with wearable device 104 regularly during operation. For example, the other wireless-enabled devices may include a mobile computing device (e.g., smart phone, tablet computing device, personal digital assistant (PDA), laptop computing device, etc.) associated with the patient and communicatively coupled to wearable device 104. In some embodiments, computer system 102 can cooperate with wearable device 104 to implement a static location tracking rate, such as once per minute or once per five-second interval. Alternatively, computer system 102 and wearable device 104 can implement a dynamic location tracking rate. For example, a controller integrated into wearable device 104 can predict a current activity of a patient wearing wearable device 104—such as sleeping, sitting, walking, or running, etc.—based on outputs of motion and/or inertial sensors integrated into the wearable device. In some embodiments, when the patient is determined to be sleeping or sitting, wearable device 104 is configured to broadcast a wireless signal—which may be collected by local wireless beacons and transformed into a location of wearable device 104 by computer system 102—at a rate of once per five-minute interval. In some embodiments, when the patient is determined to be walking slowly (e.g., less than 2 miles per hour (mph), less than 5 mph, less than 10 mph, etc.), wearable device 104 is configured to broadcast a wireless signal at a rate of once per ten-second interval. As the patient's speed of motion increases, wearable device 104 may increase its broadcast rate. For example, wearable device 104 may increase its broadcast rate up to a maximum broadcast rate, which may be once per five-second interval. Furthermore, in response to an incident, such as a fall event 302, computer system 102 is configured to transmit a command to wearable device 104 (e.g., via a local wireless beacon) to increase the broadcast rate to 1 Hz.

However, wearable device 104, the wireless beacon(s), and/or computer system 102 (e.g., location data collection subsystem 112) can cooperate in any other way to determine the location of wearable device 104, and thus the patient associated with wearable device 104. Wearable device 104, the wireless beacon(s), and/or computer system 102 can repeat these processes over a time period to track the location of the patient throughout the care facility. In particular, based on the patient's determined location(s), computer system 102 can generate a time series of the patient's locations within the care facility to identify the location of the patient as a function of time. Thus, computer system 102 can extract various ADL data from movements 304, such as when and where the patient spends his/her time (or most of his/her time), the patient's physical activity level and mobility level, with whom the patient spends his/her time, and whether any of these metrics have changed, which may indicate a change in the patient's comfort level in the care facility, friend group, physical health, and/or mental health. As described herein, computer system 102 can implement similar methods and techniques to track locations of other wearable devices 104 assigned to and worn by other patients of the care facility over the same period of time or over different periods of time.

In some embodiments, mobility detection subsystem 114 is configured to determine an amount of movement of a patient wearing wearable device 104 during a time period. Mobility detection subsystem 114 may receive the location data from location data collection subsystem 112 and/or location database 132. In some embodiment, the location data includes a time series of locations of a patient. Mobility detection subsystem 114 of computer system 102 is configured, in some embodiments, to transform the location data collected by location data collection subsystem 112 into an estimation of an amount of movement, daily activities, and other motion information for a given patient wearing wearable device 104. In some embodiments, mobility detection subsystem 114 is configured to transform location data for a large number of patients of one or more care facilities into an estimation of the amount of movement, daily activities, and other motion information, for each patient. As described herein, the mobility data is also referred to herein interchangeably as ADL data. As mentioned previously, location data collection subsystem 112 is configured to track the location and motion of patient resident in relation to a known floorplan (e.g., floorplan 210) or other map of the care facility (e.g., care facility 200) over time. By analyzing the location and motion of each patient in relation to the floorplan of the care facility, mobility detection subsystem 114 is able to estimate and/or categorize the activity being performed by the patient at any given time. For example, if the patient is located within an area corresponding to the bed of the patient within the patient's room (e.g., room 212a), mobility detection subsystem 114 may estimate that the patient is engaging in sedentary behavior and/or sleeping. Additionally or alternatively, mobility detection subsystem 114 is configured to cross-reference the location data and the motion data with inertial and biometric data of the patient to categorize the current activity of the patient. For example, if the patient is located within an area corresponding to the bed of the patient and wearable device 104 of the patient is reporting inertial data consistent with sleeping behavior, mobility detection subsystem 114 can categorize the activity as “sleeping time.”

In some embodiments, mobility detection subsystem 114 utilizes an ADL model to transform location data and motion data collected from each wearable device 104 of a corresponding patient into an ADL profile for that patient. For instance, mobility detection subsystem 114 can obtain an ADL model from model database 142. The ADL model can include any type of classifier for identifying each ADL and the amount of time the patient spends performing each ADL. For example, the ADL model may be configured to classify motion of a patient during a certain time period as being walking motion or motion representative of walking behavior based on the motion data recorded by wearable device 104. In some embodiments, the ADL model is a conditional model that records the patient as performing a particular ADL when a set of conditions corresponding to that ADL are satisfied based on the location and motion data of the patient. For example, the ADL may include a geofence around the toilet of a patient in the care facility, and the ADL may classify any time spent within the geofence as “toileting” activity. Alternatively, the ADL model can be a supervised learning algorithm that classifies patient behavior based on labels provided by providers. For example, a provider associated with provider client device 106 may classify an ADL as being an exercise or movement activity based on the provider knowingly facilitating a physical therapy for the patient at a certain time.

In some embodiments, mobility detection subsystem 114 is also configured to calculate secondary ADL metrics indicating various aspects of each patient's quality of life. For example, mobility detection subsystem 114 is configured to compute metrics for sleep quality, mobility, and/or sociability, based on observed location and motion data for a patient. However, alternatively, the secondary ADL metrics are computed using other components of computer system 102, wearable device 104, and/or provider client device 106, and mobility detection subsystem 114 is configured to record ADL data related to mobility of the patient (e.g., mobility data).

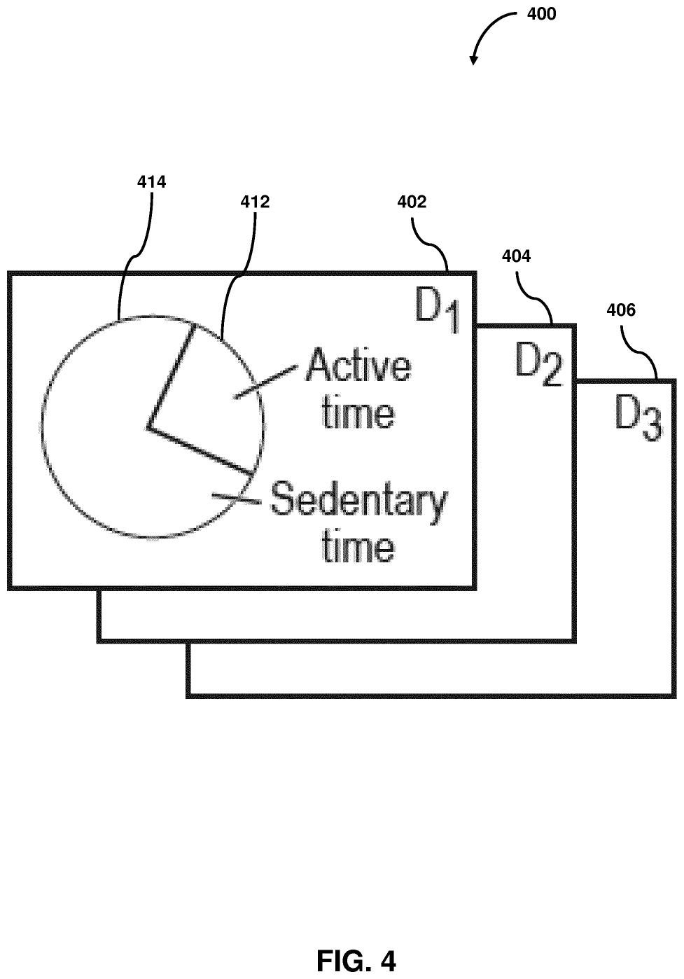

In some embodiments, mobility detection subsystem 114 is configured to categorize each moment in the time series of location data, inertial data, and/or biometric into an ADL, thereby estimating a time series of activities of the patient. However, because the activities of a patient can be highly dependent on the time of day (and often the time of week), mobility detection subsystem 114 is operable to represent the ADL data as a sequence of daily totals of the ADLs of the resident. As an example, with reference to FIG. 4, ADL data 400 may include entries 402-406, each of which is associated with a time period during which location data and mobility data were captured for a patient wearing wearable device 104. For instance, entry 402 illustrates an amount of movement 412 and an amount of sedentary time 414 exhibited by a patient during a first time period D1. Amount of movement 412, which may also be referred to herein interchangeably as “active time,” indicates a total amount of time of first time period D1 during which ADL data 400 classified the patient as moving. Amount of sedentary time 414, which may also be referred to herein interchangeably as “sedentary time,” indicates a total amount of time of first time period D1 during which ADL data 400 classified the patient as being sedentary. In some embodiments, amount of sedentary time 414 includes sleep time of the patient. Alternatively, sleep time may be excluded from entries 402-406, as ADL data 400 may extract that data or represent sleep data in alternative representations dedicated to sleep analysis. Entries 404 and 406 illustrate an amount of movement and an amount of sedentary time of the patient during a second time period D2 and a third time period D3, respectively.

In some embodiments, mobility detection subsystem 114 is configured to format the ADL data (e.g., ADL data 400) as a vector array, wherein each entry in the array is a vector representing the various ADLs estimated for a particular patient for a given time period (e.g., one 24-hour time period). Mobility detection subsystem 114 is capable of representing the ADL data as a series of “daily entries,” wherein each “daily entry” corresponds to an entry in an array. Additionally or alternatively, mobility detection subsystem 114 is capable of representing the ADL data on a per-week basis (e.g., the time period is seven consecutive 24-hour time periods) to account for fluctuations in patient behavior on a weekly schedule by averaging the ADL data recorded for each day over each week represented in the data. However, mobility detection subsystem 114 is capable of representing the ADL data in any suitable data format. Furthermore, the time period with which the ADL data is represented may be less than one day (e.g., hourly, every 6 hours, every 12-hours), multiple days, weekly, bi-weekly, monthly, etc.

In some embodiments, medication data retrieval subsystem 116 is configured to retrieve medication data indicating a set of medications taken or prescribed to be taken by a patient during a given time period. Medication taken by a patient can be noted in an electronic health record of the patient by a provider. For example, in response to a provider (e.g., a doctor, nurse, medical technician, etc.) administering one or more medications, the provider can annotate and/or create an electronic health record to indicate that the one or more medications were administered to the patient. Alternatively, medication prescribed to be taken by a patient may include an indication from one or more health records of the patient that a provider (e.g., doctor, nurse, etc.) prescribed a medication or combination of medications for the patient to take during a given time period and/or with a given frequency of administration (e.g., daily, twice a day, with food, etc.). Medication data retrieval subsystem 116 may obtain medication data for a patient by accessing health record database 134. For example, a patient identifier 12345 associated with a patient lmay be used to query health record database 134 to retrieve one or more health records associated with that patient ID. Medication data retrieval subsystem 116 is configured to extract information indicating a set of medications identified from the health records as having been prescribed to the patient during the first time period. Furthermore, medication data retrieval subsystem 116 can determined, for each medication in the set of medications, a sub-period of the first time period during which the medication was prescribed to be taken by the patient. For example, a first medication M₁ may be prescribed to be taken by the patient during a first sub-period of a first time period. As another example, both first medication M₁ and a second medication M₂ may be prescribed to be taken by the patient during the first sub-period of the first time period. Health record database 134 is configured to store health records associated with one or more patients. In some embodiments, the health records may be electronic health records (EHR). Therefore, medication data retrieval subsystem 116 is configured to retrieve EHR data stored in health record database 134, which may be used to obtain the medication data for a patient. In some embodiments, the EHR data may be accessed via an EHR application. Medication data retrieval subsystem 116 is configured, in some embodiments, to access the health record database 134 to obtain the medication data pertaining to each of the patients from a care facility or amongst a set of care facilities. In some embodiments, health record database 134 includes multiple databases associated with multiple care facilities, and medication data retrieval subsystem 116 is configured to access each instance of health record database 134 corresponding via a particular EHR application for a given care facility.

In some embodiments, a provider application resident on provider client device 106 is also configured to function as an EHR application for one or more care facilities of the set of care facilities. For instance, the EHR application for computer system 102 and the care provider application for provider client device 106 may access the same database or set of databases to perform their respective tasks. Thus, medication data retrieval subsystem 116 of computer system 102 can access EHR data freely from the care provider application/EHR application database that is controlled by the same administrator. However, computer system 102 is operable to access EHR data for a set of residents of a care facility in any other way, such as manual data entry by a care provider.

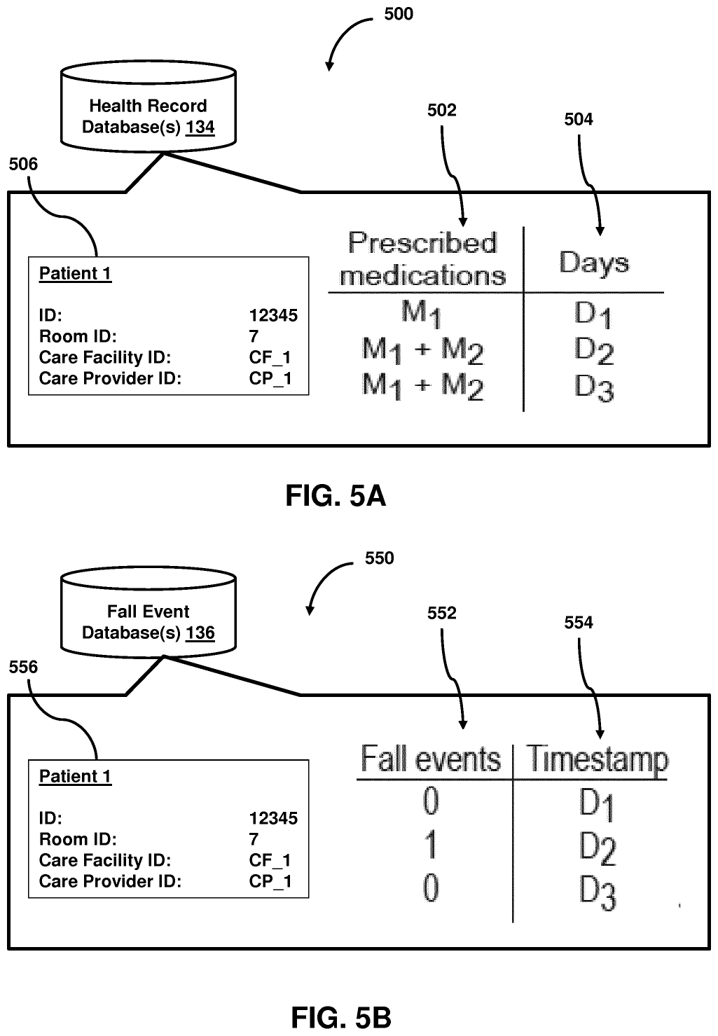

In some embodiments, upon accessing the EHR data from health record database 134, medication data retrieval subsystem 116 is configured to normalize the EHR data such that the EHR data is in a usable format for labelling the ADL data and a set of fall events, as detailed below. As an example, medication data retrieval subsystem 116 is configured to reformat the EHR data into a series of daily entries, wherein each daily entry includes identification numbers representing medications prescribed to the resident on the corresponding day. As an example, with respect to FIG. 5A, EHR data 500 is configured to be accessed from health record database 134. EHR data 500 may include medication data associated with a given patient. For example, medication data includes one or more medications 502 taken or prescribed to be taken by a patient (e.g., patient 1) during time periods 504. In some embodiments, EHR data 500 includes one or more health records associated with a patient (e.g., patient 1), and includes patient identification information 506. Patient identification information 506 includes, for example, a patient identifier for the patient (e.g., patient ID 12345), a room identifier of the room (e.g., room 212a) that the patient resides (e.g., room ID 7), a care facility identifier (e.g., CF_1), a care provider identifier (e.g., CP_1), and/or other information. Medications 502 indicate which medication or medications taken or prescribed to be taken by the patient during time periods 504. For example, during time period D1, medication M₁ is taken or prescribed to be taken by patient 1; during time period D2, medications M₁ and M₂ are taken or prescribed to be taken by patient 1; and during time period D3, medications M₁ and M₂ are taken or prescribed to be taken by patient 1. In some embodiments, EHR data 500 includes names of each of medications 502 taken or prescribed to be taken by patient 1. Alternatively, EHR data 500 may include additional information about the medications, such as a prescribed dosage, dosage schedule, and/or a chemical composition of the medications. Furthermore, EHR data 500 may include other clinical data pertaining to each of the patients such as health conditions, vital signs, recent medical treatments, etc. For example, EHR data 500 may include a clinical status of one or more patients, where the clinical status indicates one or more previous and/or current medical diagnoses of the patient, vital signs of the patient, and the like.

In some embodiments, fall event detection subsystem 118 is configured to retrieve fall event data from wearable device 104 and/or fall event database 136. The fall event data may include a set of fall events experienced by a patient during a particular time period. In some embodiments, wearable device 104 is configured to detecting a set of fall events involving a patient. In some embodiments, fall events (e.g., fall events and/or calls for assistance from a resident) may be detected by wearable devices 104 worn by each patient amongst the set of care facilities. In some embodiments, fall events may be detected by wearable devices 104 via wireless beacons located in and around each care facility. Fall event detection via a wearable device, such as wearable device 104, is described in commonly-assigned U.S. patent application Ser. No. 15/627,186, entitled “METHOD FOR DETECTING AND RESPONDING TO FALLS BY RESIDENTS WITHIN A FACILITY,” filed on Jun. 19, 2017, and which issued as U.S. Pat. No. 10,226,204 on Mar. 12, 2019, the disclosures of which are incorporated herein by reference in their entireties. In some embodiments, fall event detection subsystem 118 is configured to calculate a frequency of incidents, such as fall events, in which a particular patient is involved over a period of time (e.g., one week, one month, one year, etc.). In some embodiments, fall event detection subsystem 118 is configured to detect a set of fall events based on data captured by wearable device 104. A fall event, as described herein, is any incident with which a particular patient is determined to have fallen. A fall event may include accidental falls, intentional falls, etc.

Wearable device 104 is configured to execute a compressed fall detection model for implementation on a compact device and characterized by a low rate of false-negative fall event outputs. In some embodiments, fall event detection subsystem 118 is configured to execute a complete fall detection model requiring greater memory than the compressed fall detection model also characterized by greater precision. Wearable device 104 can therefore selectively and intermittently communicate with computer system 102 when a fall event is detected locally in order to prompt fall event detection subsystem 118 of computer system 102 to confirm the fall event and to record the fall event (e.g., within memory 108 and/or within fall event database 136), thereby limiting power consumption by wearable device 104 while maintaining access to a relatively high-precision fall detection model.

In some embodiments, wearable device 104 includes one or more three-axis gyroscopes, one or more three-axis accelerometers, a compass, a barometer, a skin temperature sensor, a heart sensor, and/or one or more inertial sensors. A processor of wearable device 104 is capable of being configured to pass data from one or more of these sensors into the fall detection model in order to detect a fall event. Additionally, memory of wearable device is capable of being configured to store sensor data (e.g., data packets) in a buffer of limited duration (e.g., one minute, three minutes). Wearable device 104 can also include one or more rechargeable batteries to power the foregoing elements, and/or a wireless communication module that broadcasts fall event data to local wireless hubs of a care facility when the processor detects a fall event and that broadcasts beacons to local wireless hubs throughout the care facility to enable location data collection subsystem 112 and/or fall event detection subsystem 118 of computer system 102 to track the location of the wearable device 104—and therefore the patient—throughout the care facility over time. The processor can sample the set of internal sensors at a single static sampling rate or at different and/or dynamic sampling rates. For example, the processor can sample the gyroscope and accelerometer at a sampling rate of 100 Hz, sample the heart rate sensor once per minute (1/60 Hz), and sample the skin temperature sensor once per three-minute interval (1/300 Hz).

In some embodiments, fall event detection subsystem 118 is configured to timestamp each fall event in the set of fall events or receive timestamps for each fall event in the set of fall events. For example, upon detecting a fall event, fall event detection subsystem 118 is configured to associate a time, sub-period of a time period, and/or a time period, with the fall event (i.e., time-stamps the fall event) and an identification of the patient who fell based on the NMS. Fall event detection subsystem 118 may then cause the fall event data to be stored in fall event database 136 (e.g., for labelling according to EHR data for the patient). As an example, with reference to FIG. 5B, fall event data 550 may include a number of fall events 552 detected for one of time periods 554, as well as patient identification information 556. Patient identification information 556 includes, for example, a patient identifier for the patient (e.g., patient ID 12345), a room identifier of the room (e.g., room 212a) that the patient resides (e.g., room ID 7), a care facility identifier (e.g., CF_1), a care provider identifier (e.g., CP_1), and/or other information. In some embodiments, patient identification information 556 is the same or similar to patient identification information 506, and the previous description applies. As an example, number of fall events 552 and time periods 554 may indicate that during first time period D1, 0 fall events were detected by wearable device 104 for patient 1; during second time period D2, 1 fall event was detected by wearable device 104 for patient 1; and during third time period D3, 0 fall events were detected by wearable device 104 for patient 1.

In some embodiments, model training subsystem 120 is configured to generate training data for training a prediction model. The prediction model may be capable of being used to estimate a dependency between one or more medications taken or prescribed to be taken by a patient and an increase in risk of the patient experiencing a fall event. The prediction model may also be capable of being used to estimate a dependency between one or more medications taken or prescribed to be taken by a patient and an increase or decrease in mobility of the patient. In some embodiments, model training subsystem 120 generates the training data in response to receiving a request from provider client device 106 for estimating one or both of the aforementioned dependencies.

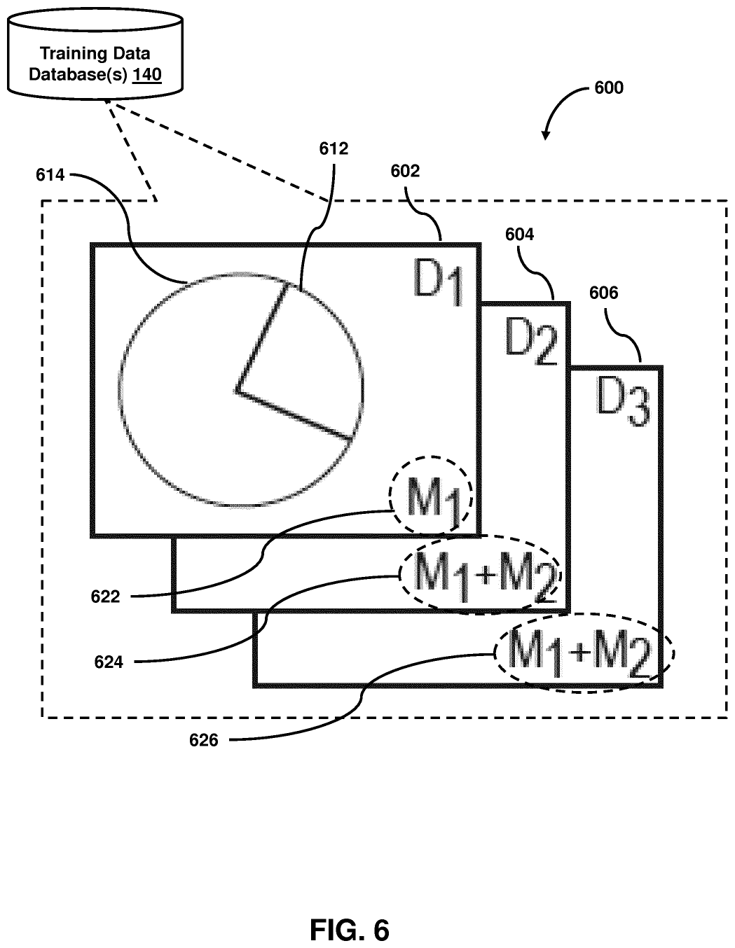

In some embodiments, the training data to be generated by model training subsystem 120 includes labeled mobility data. The labeled mobility data includes the mobility data, previously detected by mobility detection subsystem 114, labeled based on the medication data previously retrieved by medication data retrieval subsystem 116. For each entry of the mobility data, the entry may be labeled with a subset of the set of medications, the subset representing one or more medications taken or prescribed to be taken by the patient during a time period associated with the given entry. As an example with reference to FIG. 6, labeled mobility data 600 includes entries 602-606, each of which is associated with a time period during which location data and mobility data were captured for a patient wearing wearable device 104. Similar to entries 402-406 of ADL data 400 illustrated by FIG. 4, entry 602 illustrates an amount of movement 612 and an amount of sedentary time 614 exhibited by a patient during a first time period D1. Furthermore, entry 602 may also be labeled by training data subsystem 120 based on the medication data to indicate a medication or medications taken or prescribed to be taken by a patient during time period D1. For instance, entry 602 includes a label 622 indicating that medication M₁ was taken or prescribed to be taken during first time period D1 based on EHR data 500. Entry 604 includes a label 624 indicating that medications M₁ and M₂ were taken or prescribed to be taken during second time period D2 based on EHR data 500. Furthermore, entry 606 includes a label 626 indicating that medications M₁ and M₂ were taken or prescribed to be taken during third time period D3 based on EHR data 500.

Generally, ADL data (e.g., ADL data 400) can be expressed on a daily basis such that each day within the span of ADL data has an entry describing activities of the patient on that particular day. Thus, computer system 102 can detect concurrency between daily entries of ADL data and daily entries of EHR data and label ADL data according to the identification. For example, if computer system 102 detects that ADL data for a particular patient contains a daily entry corresponding to the ninth of September in 2018 and the EHR data also contains a daily entry corresponding to the ninth of September in 2018, model training subsystem 120 of computer system 102 may be configured to label the ADL data according to the identifications of medications taken or prescribed to be taken as indicated within the EHR daily entry. In some embodiments, the above described labelling process is capable of being repeated for each patient of each care facility and for each day for which both ADL data and EHR data are available.

In some embodiments, the training data to be generated by model training subsystem 120 additionally or alternatively includes labeled fall event data. The labeled fall event data includes the fall event data, previously detected by fall event detection subsystem 118, labeled based on the medication data previously retrieved by medication data retrieval subsystem 116. For each fall event in the set of fall events of the fall event data, the fall event may be labeled with a subset of the set of medications, the subset representing medications taken or prescribed to be taken by the patient during a time period associated with the fall event. For example, model training subsystem 120 may label fall events represented by fall event data 550 with medication data based on EHR data 500. In some embodiments, model training subsystem 120 is configured to detect concurrency between each fall event and an entry of EHR data 500 based on a timestamp associated with each fall event and the time period corresponding to the entry of EHR data 500. However, model training subsystem 120 is capable of formatting or representing labeled fall event data in any other way such that concurrency between the various types of data is detectable.

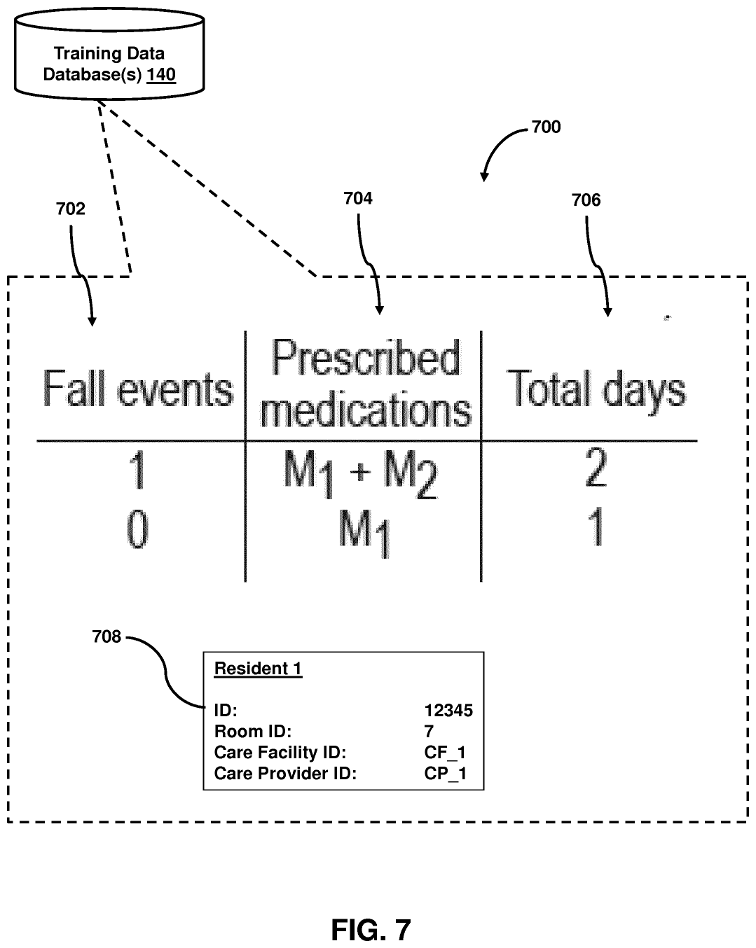

As an example, with reference to FIG. 7, labeled fall event data 700 may be generated by model training subsystem 120 and stored in training data database 140. Labeled fall event data 700 includes a number of fall events 702, prescribed medications 704, and total time 706 with which the medications were prescribed. For example, when medications M₁ and M₂ were taken or prescribed to be taken by a patient, such as during second time period D2 and third time period D3, 1 fall event was detected. However, when only medication M₁ was taken or prescribed to be taken, such as during first time period D1, 0 fall events were detected. Labeled fall event data 700 may further include patient identification information 708. Patient identification information 708 includes, for example, a patient identifier for the patient (e.g., patient ID 12345), a room identifier of the room (e.g., room 212a) that the patient resides (e.g., room ID 7), a care facility identifier (e.g., CF_1), a care provider identifier (e.g., CP_1), and/or other information. In some embodiments, patient identification information 708 is the same or similar to patient identification information 506, and the previous description applies.

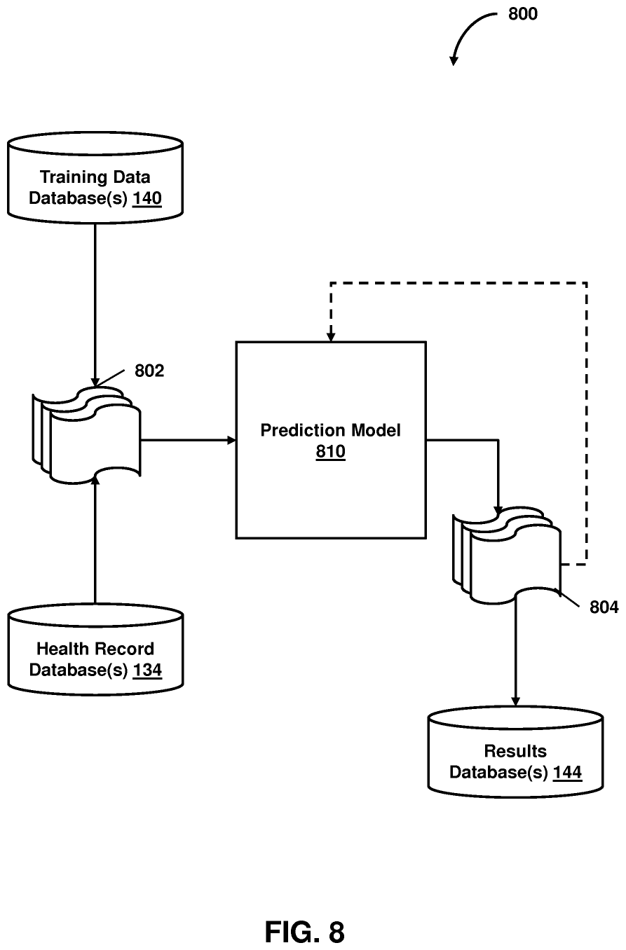

FIG. 8 shows an example system 800 including an example predication model 810, in accordance with various embodiments. In system 800, prediction model 810 is configured to take inputs 802 and provide outputs 804. In some embodiments, outputs 806 are fed back to predication model 810 as an input to train predication model 810 (e.g., alone or in conjunction with user indications of the accuracy of outputs 806, labels associated with the inputs, or with other reference feedback information). In some embodiments, predication model 810 may update its configurations (e.g., weights, biases, or other parameters) based on its assessment of its prediction (e.g., outputs 806) and reference feedback information (e.g., user indication of accuracy, reference labels, or other information). In some embodiments, where predication model 810 is a neural network, connection weights may be adjusted to reconcile differences between the neural network's prediction and the reference feedback. Some embodiments include one or more neurons (or nodes) of the neural network requiring that their respective errors are sent backward through the neural network to them to facilitate the update process (e.g., backpropagation of error). Updates to the connection weights may, for example, be reflective of the magnitude of error propagated backward after a forward pass has been completed. In this way, for example, the predication model 810 may be trained to generate better predictions. In some embodiments, prediction model 810 is a supervised or unsupervised machine learning model, a statistical model, a non-statistical model, or another analytics model.

In some embodiments, prediction model 810 is configured to estimate a dependency between one or more medications in the set of medications and reduction or increase in mobility for a resident based on the labelled mobility data. In some embodiments, prediction model 810 is configured to estimate a dependency between one or more medications and an increase in a risk of a patient experiencing a fall event based on the labelled fall event data. Model training subsystem 120 may utilizes prediction model, a statistical model, or other analytics model, such as a drug interaction model, to determine statistical dependencies between particular medications taken or prescribed to a patient or patients and outcomes in the form of mobility changes and fall events. In some embodiments, a drug interaction model includes performing an ANOVA test across the medications prescribed to a set of patients of a care facility to determine if there is a statistically significant difference (i.e., a dependency) between ADL outcomes for patients taking or prescribed to take a particular medication or a combination of medications. Alternatively or additionally, the drug interaction model can include a Kruskall-Wallis test to calculate dependencies between combinations of medications taken or prescribed to be taken by one or more patients and ADL outcomes. Furthermore, model training subsystem 120 can perform the same set of tests to calculate a statistical dependency between the frequency of fall events and the combinations of medications taken or prescribed to taken by a resident.

In some embodiments, prediction model 810 includes a trained supervised learning model based on labelled ADL data and/or labelled fall events to predict ADL outcomes and/or frequency of fall events for a patient based on the combination of medications taken or prescribed to be taken by a patient. In some embodiments, inputs 802 include training data is generated based on labeled mobility data 600. For example, the training data may include labeled mobility data 600. In some embodiments, training data may be generated based on labeled fall event data 700. For example, the training data may include labeled fall event data 700. In some embodiments, labeled mobility data 600 and labeled fall event data 700 are both configured to be stored in training data database 140. In some embodiments, the training data, which is generated based on labeled mobility data 600, labeled fall event data 700, or both, is stored in training data database 140. Inputs 802 may include the training data and/or be formed based on the training data.

Upon receiving a request to train prediction model 810, or cause prediction model 810 to be trained, training data is capable of being obtained from training data database 140. Inputs 802 are configured to be generated based on the training data. Depending on the dependency to be estimated by the prediction model, a particular instance or type of training data may be obtained. For example, if prediction model 810 is to determine the dependency between one or more medications and an increase in a risk of a patient experiencing a fall event, inputs 802 for prediction model is generated based on the training data including labeled fall event data 700 may be retrieved from training data database 140. In some embodiments, additional EHR data for inclusion in training the prediction model 810 (e.g., a supervised learning model) may also be retrieved. For example, model training subsystem 120 may access EHR data pertaining to medical conditions or the clinical status of each patient from health record database 134. The clinical status can indicates one or more previous and/or current medical diagnoses of the patient, vital signs of the patient, and the like. By including additional EHR data in prediction model 810 (e.g., a supervised learning model), model training subsystem 120 can facilitate prediction model 810 providing improved estimates of fall event risk and ADL outcomes. In some embodiments, model training subsystem 120 is configured to periodically reevaluate prediction model 810 to update the statistical dependencies between combinations of medications and resident outcomes. Alternatively, model training subsystem 120 may update prediction model 810 whenever computer system 102 determines additional data associated with a patient or set of patients is obtained.

In some embodiments, prediction model 810 is configured to utilize mobility data (e.g., in-room movement time plus out-of-room movement time) from the ADL data and calculates the statistical dependency of a patient's mobility on one or more medications instead of calculating the statistical dependency of all ADL data on the one or more medications. However, prediction model 810 can detect statistical dependency between combinations of prescription medications and ADL outcomes and fall events in any other way, and the aforementioned should not be construed as limiting.

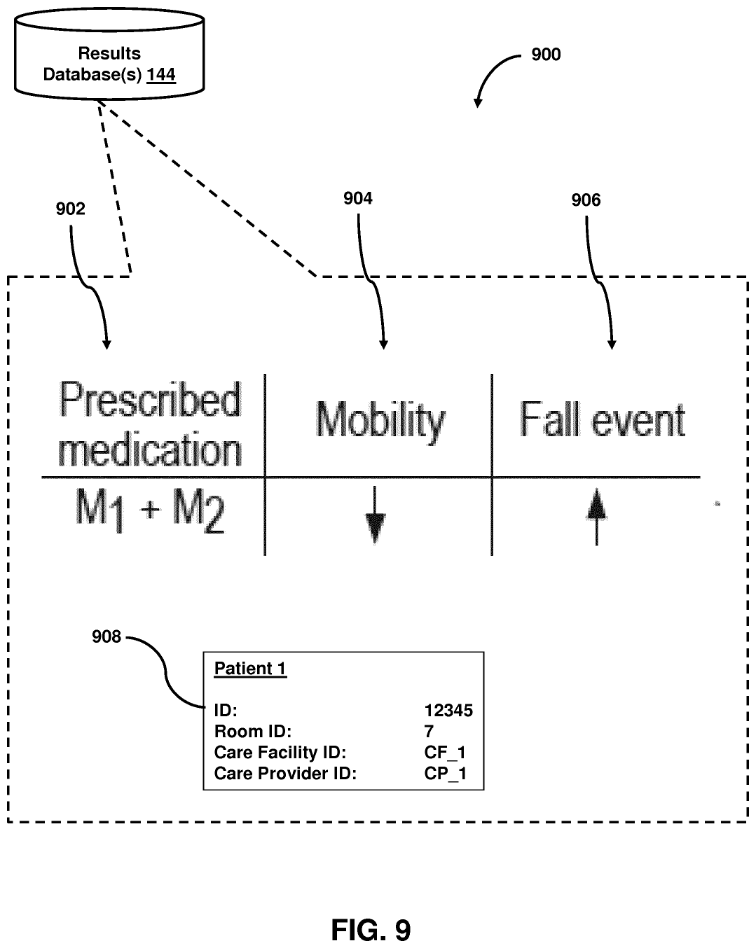

In some embodiments, outputs from prediction model 810 are configured to be stored in results database 144. The outputs may include results associated with various medications and combinations of medications. Each result is capable of indicating which, if any, adverse medication effects a patient prescribed a given medication or combination of medications is at risk to experience. As an example, with reference to FIG. 9, result 900 is stored within results database 144, and indicates that for a combination of medications 902 taken or prescribed to be taken by a patient, adverse medication effects 904 and 906 may be experienced by the patient. Furthermore, result 900 is configured to include patient identification information 908. For example, patient identification information includes a patient identifier for the patient (e.g., patient ID 12345), a room identifier of the room (e.g., room 212a) that the patient resides (e.g., room ID 7), a care facility identifier (e.g., CF_1), a care provider identifier (e.g., CP_1), and/or other information. In some embodiments, patient identification information 908 is the same or similar to patient identification information 506, and the previous description applies.

If a new or existing patient of a care facility takes or is prescribed to take a new medication or combination of medications, inputs 802 may, in some embodiments, correspond to the medication data. For instance, medication data indicating one or more medications taken or prescribed to be taken is retrieved from health record database 134. The medication data is capable of being retrieved in response to a patient being added to a portion of health record database 134 for a care facility, indicating that the patient is new. Alternatively, the medication data is capable of being retrieved in response to a detection that a new electronic health record for a patient has been added to health record database 134. The medication data is capable of being used to generate inputs 802, which may be provided to prediction model 810. In an example, inputs 802 corresponding to the medication data for a new patient or an existing patient prescribed a new medication or medications may be provided to a trained instance of prediction model 810. For example, subsequent to prediction model 810 being trained, inputs 802 corresponding to the aforementioned medication data is provided to the trained instance of prediction model 810.

The trained instance of prediction model 810 is configured to provide an output or outputs 804, indicating a risk that a set of medications can cause the patient to experience adverse medication effects. For instance, outputs 804 may include a risk value. In some embodiments, if the risk value is determined to be greater than or equal to a threshold risk value, then a warning notification may be generated. For example, warning detection subsystem 122 may cause a warning notification to be generated if a risk value output by a trained instance of prediction model 810 is greater than or equal to a threshold risk value. As an example, the risk value output by the trained instance of prediction model 810 (e.g., outputs 804) may be a numerical value (e.g., a number between 0 and 100), and the threshold risk value may also be a numerical value (e.g., a number greater than 50, a number greater than 60, a number greater than 70, etc.).

In some embodiments, result 900 is an output (e.g., outputs 806) of a prediction model after some or all of training. As an example, result 900 indicates that combination of medications 902 includes medications M₁ and M₂. Combination of medications 902 may cause a first adverse medication effect 904, which includes an indication that the combination of medications taken or prescribed to be take by a patient can cause a decrease in mobility for the patient. Combination of medications 902 additionally or alternatively may cause a second adverse medication effect 906, which includes an indication that the combination of medications taken or prescribed to be taken by a patient can cause an increase in risk of the patient experiencing fall events. Alternatively, first adverse medication effect 904 may cause an increase a mobility, and second adverse medication effect 906 may cause a decrease in risk of the patient experiencing fall events. Furthermore, additional or alternative adverse medication effects are possible, including, but not limited to, an increase or decrease in body temperature, pulse, blood pressure, respirations, appetite, sociability, eyesight, a combination thereof, or other possible side effects.

In some embodiments, warning detection subsystem 122 is configured to generate and/or provide, or cause to be provided, a warning notification to a provider. The warning notification may indicate that a particular patient of a care facility has an increased likelihood of experiencing one or more adverse medication effects. The warning notification may be provided to provider client device 106, which is capable of displaying visual alerts and/or information to a provider, audible alerts and/or information to the provider, haptic alerts to the provider, a combination thereof, or other alerts and/or information.

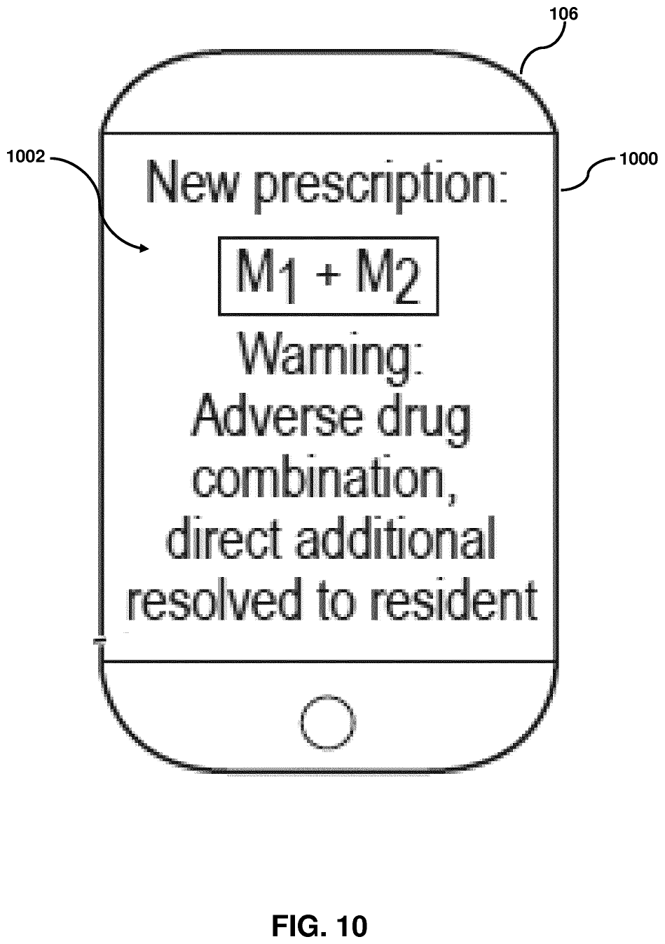

In some embodiments, in response to detecting a new medication or combination of medications being taken or prescribed to be taken by a patient (new or existing) at a care facility, a warning notification can be generated and provided to a care provider application operating on provider client device 106. The warning notification may direct a provider operating provider client device 106 to provide a check-in with the new patient, alert other providers to check-in with the new patient, cause additional care to be provided to the new resident, a combination thereof, and/or other possible assistance. As an example, with reference to FIG. 10, provider client device 106 is configured to render a graphical user interface (GUI) 1000 on a display screen 1000. GUI 1002 may be configured to output a warning notification to a provider associated with provider client device 106 that indicates that a new patient has been provided a combination of medications that can cause an adverse medication effect. In some embodiments, GUI 1000 may also provide an option for the provider to facilitate an action to occur to provide assistance to the patient indicated by the warning notification. For example, GUI 1002 may include an input, such as a physical or virtual button that may be pushed. As another example, GUI 1002 in connection with the care provider application resident on provider client device 106, may be configured to detect an input, such as a finger swipe, click, tap, and the like. In some embodiments, detection of the input may certain actions to be performed. For example, upon detection of the input, care provider application alone or in communication with warning detection subsystem 122 can cause another provider or set of providers determined as being proximate to a location of the patient to be alerted so as to check in on a health status of the patient. As indicated previously, location data collection subsystem 112 is configured to obtain location data indicating a location of each provider of a care facility as well as of a patient residing at the care facility. Using the location data, providers that are determined as being proximate to the patient (e.g., within a threshold distance of the location and/or area of the patient), each provider client device 106 of the proximate providers may receive the warning notification, as well as, or alternatively additional information (e.g., a name, room number, floor number, adverse medication effect, etc.) for potentially treating the adverse medication effect if currently being experienced by the patient.

In some embodiments, warning detection subsystem 122 is configured to execute a drug interaction warning system that monitors EHR data of current patients of a set of care facilities. If, or upon, detecting a patient taking or being prescribed to take a particular combination of medications that have been determined to potentially cause one or more adverse medication effects, warning detection subsystem 122 can generate and send a warning notification to a care provider application executing on provider client device 106 of a provider associated with the patient explaining the calculated risks or predicted changes in ADL outcomes or fall event frequency for the patient.

In some embodiments, model training subsystem 120 and/or warning detection subsystem 122 executes prediction model 810 or causes prediction model 810 to be executed (e.g., the drug interaction model) to predict ADL outcomes and fall event frequency for certain combination of medications. The results of the predictions for each combination of medication may be stored in results database 144. In some embodiments, warning detection subsystem 122 detects whether a new patient has taken or has been prescribed to take one or more medications included within the combination of medications whose ADL outcomes and/or fall event frequency has been predicted. For example, warning detection subsystem 122 and/or medication data retrieval subsystem 116, may detect a presence of a new prescription in a patient's EHR data, (e.g., one or more health records associated with the new patient), stored in health record database 134.

In some embodiments, warning detection subsystem 122 is configured to obtain the predicted ADL outcomes and fall event frequency for a new combination of medications prescribed to a new or existing patient of a care facility. Warning detection subsystem 122 is further configured to compare the predicted outcomes and fall event frequency to the current ADL outcomes and/or fall event frequency of the patient given the set of medications currently prescribed to the new patient. The set of medications currently prescribed to the new patient may be determined based on one or more health records of the new patient, which may be retrieved from health record database 134. If the difference between the current and predicted outcomes is greater than a threshold, warning detection subsystem 122 is operable to generate and provide a warning notification describing the predicted changes to a care provider application associated with provider client device 106. For example, if the outcome relates to a number of fall events experienced by a patient increasing by a threshold number of fall events (e.g., 1 fall event, 2 fall events, 5 fall events, etc.), the warning notification may indicate that the patient is at risk of experiencing an increase in fall events. As another example, if the outcome relates to an amount of movement (e.g., active time) of the patient decreasing by a threshold amount of time with respect to an average amount of movement of the patient, the warning notification may indicate that the patient is at risk of experiencing a decrease in mobility. The threshold amount of time, which can also be referred to as a threshold amount, may be a number of minutes, hours, percentage of a total amount of movement of a patient during a given time period, etc. Therefore, if the amount of movement changes by more than a particular number of minutes, hours, or percentage of the total amount of movement of the patient, this may indicate that the patient is at risk of experiencing one or more of the adverse medication effects. In some embodiments, the average amount of movement may be computed by averaging an amount of movement experienced by the patient during a previous time period or time periods. For example, the average amount of time may be represented as a number of minutes, hours, etc., that the patient was detected as being active during a particular time period. As another example, the average amount of time may be represented as a percentage of time of the day that the patient was determined as being moving. Additionally or alternatively, warning detection subsystem 122 is operable to generate and provide a warning notification characterizing the predicted change in ADL outcomes and fall event risk for a patient as positive or negative. Additionally, the warning notification can also indicate the degree of the predicted change (e.g., a severe risk of expiring a fall event).

In some embodiments, warning detection subsystem 122 is configured to provide the warning notification indicating the potential adverse medication effects (e.g., predicted ADL outcomes and/or fall event frequency) when prediction model 810 (e.g., the drug interaction model) has calculated that the current set of medications taken or prescribed to be taken by the patient. In some embodiments, the warning notification includes information explaining a statistical significance and/or a statistically significant difference in ADL data and fall event frequency. For example, the warning notification may include an indication that a particular patient has a 80% chance increased fall event experiences. In some embodiments, warning detection subsystem 122 can directly report the predicted ADL outcomes and fall event risk to providers (e.g., via the care provider application executing via a corresponding provider client device 106) each time a new medication or medications is/are added to a patient's health records, thereby providing consistent predictions of ADL outcomes and fall event risk for consideration by providers.

In some embodiments, warning detection subsystem 122 is configured to continuously monitor the ADL outcomes and fall event frequency of each patient and compare these data to the predicted ADL outcomes and fall event frequency for the patient given the current set of medications taken or prescribed to be taken by the patient, as well as any other information included by the health records of the patient. If there is a significant difference between the predicted ADL outcomes and fall event risk, warning detection subsystem 122 is configured to generate and provide the warning notification indicating that the ADL data of a patient does not match the predicted ADL outcomes and that further investigation may be required. For example, if a medication is predicted to cause a decrease in mobility for a patient, computer system 102 (e.g., using mobility detection subsystem 114 and/or warning detection subsystem 122) can access mobility data of the patient from mobility database 134 since taking or being prescribed to take the new medication. If the recent mobility data indicate that the patient's mobility has increased, then warning detection subsystem 122 can generate and provide the warning notification to a care provider application executing via provider client device 106, the warning notification indicating that the patient may not be taking his/her medication(s) or that the patient experiencing an adverse medication effect (e.g., a side effect) to the new medication.

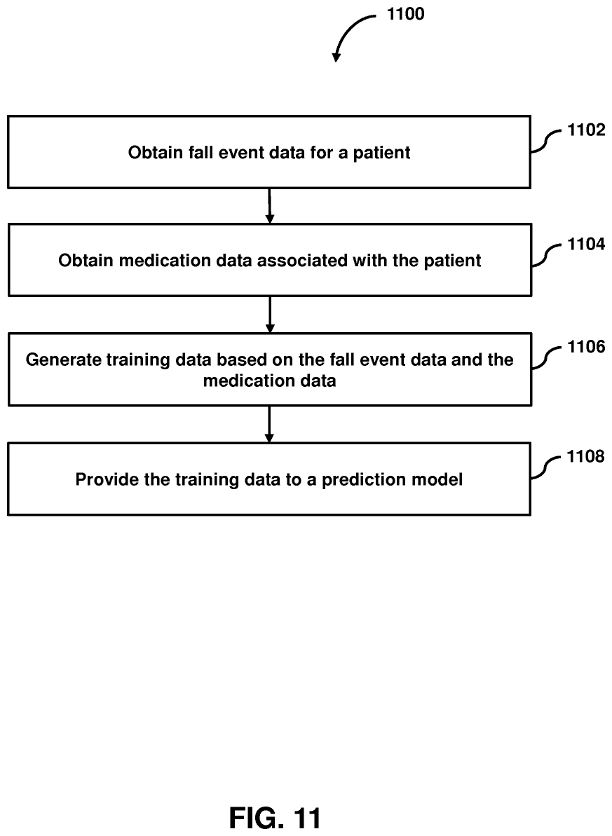

FIG. 11 shows an example method 1100 for facilitating training of a prediction model, in accordance with various embodiments. In an operation 1102, fall event data for a patient is obtained. The fall event data may be obtained from a wearable device, such as wearable device 104, which is configured to be worn by a patient of a care facility. In some embodiments, the fall event data includes a set of fall events experienced by the patient during a first time period. For example, the fall event data may indicate that the patient experienced N fall events during a 24-hour time period. As another example, the fall event data may include raw data signals from one or more motion sensors resident on wearable device 104, and a determination may be made from the fall event data as to whether the raw data signals indicate a fall event or fall events occurred during a given time period. In some embodiments, a fall detection model is configured to identify false fall events from candidate fall events detected by wearable device 104 during the first time period. Upon identifying the false fall events, one or more true fall events can be determined. In some embodiments, operation 1102 is performed by a subsystem that is the same or similar to fall event detection subsystem 118.

In an operation 1104, medication data associated with the patient is obtained. The medication data may be obtained from health record database 134. In some embodiments, one or more health records associated with the patient are retrieved from health record database 134. The one or more health records may be analyzed to extract the medication data. The medication data, for example, may indicate a set of medications taken or prescribed to be taken by the patient during the first time period. The medication data may further indicate a sub-period within the first time period during which one or more medications (e.g., one or more medications of the set of medications), are taken or prescribed to be taken by the patient. In some embodiments, operation 1104 is performed by a subsystem that is the same or similar to medication data retrieval subsystem 116.

In an operation 1106, training data is generated based on the fall event data and the medication data. The training data may include the fall event data and the medication data. For example, the training data may include a number of fall events experienced by the patient during the time period, as well as the set of medications taken or prescribed to be taken by the patient during the first time period. The training data can be stored in training data database 140, along with a timestamp indicating when the training data was generated. The timestamp may be used to determine whether a prediction model should employ the training data when being trained so as to ensure that the prediction model is trained using recently generated training data. In some embodiments, operation 1106 is performed by a subsystem that is the same or similar to model training subsystem 120.

In an operation 1108, the training data is provided to a prediction model. The training data may be provided to a prediction model, such as prediction model 810. In some embodiments, the prediction model (e.g., prediction model 810) is configured based on the training data. For example, prediction model 810 may be configured to estimate a dependency between one or more medications and an increase in a risk of a patient experiencing a fall event. In some embodiments, the prediction model is a supervised machine learning model, a neural network, or another statistical model. After being configured, the prediction model may provide results indicating the estimated dependency, and the results may be stored within results database 144. In some embodiments, operation 1102 is performed by a subsystem that is the same or similar to model training subsystem 120.

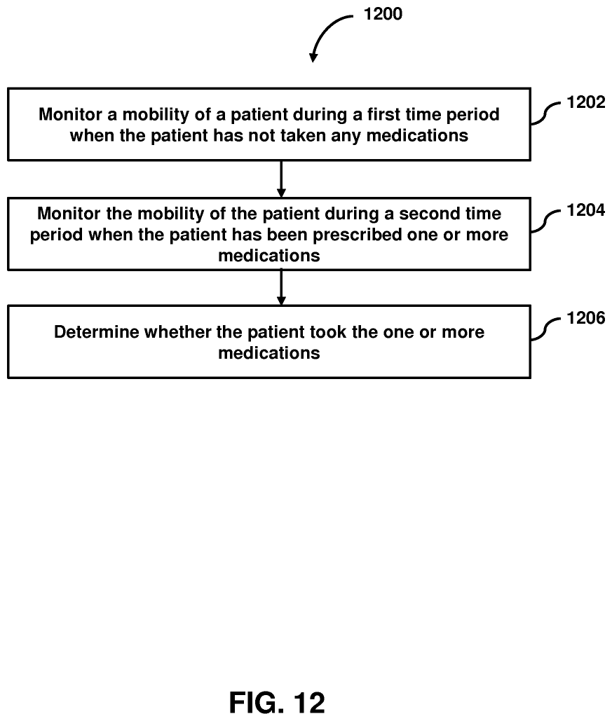

FIG. 12 shows an example method 1200 for determining whether to a patient took one or more prescribed medications, in accordance with various embodiments. In an operation 1202, a mobility of a patient during a first time period is monitored. The first time period includes a time period during which the patient has not taken, or has not been prescribed, any medications. For example, the first time period may correspond to a time period prior to the patient being a resident of a care facility, prior to being under the care and supervision of a provider (e.g., a doctor, nurse, etc.), and the like. In some embodiments, the first time period corresponds to a time period prior to the patient being prescribed one or more new medications that previously have not been, or not recently been, taken by the patient. The mobility of the patient may include an amount of movement that the patient is determined to have performed during the time period. In some embodiments, the mobility of the patient is determined by wearable device 104, which is worn by the patient during the first time period. In some embodiments, the mobility of the patient is determined based on location data obtained from wearable device 104, where the location data indicates a position of the patient during the first time period. For example, the location data may include a time series of locations of the patient, which can be used to compute an amount of movement of the patient during the first time period. Mobility data indicating the mobility of the patient during the first time period may be stored in mobility database 138, for example with an indication of the associated time period. In some embodiments, operation 1202 is performed by a subsystem that is the same or similar to location data collection subsystem 112, mobility detection subsystem 114, or a combination thereof.

In an operation 1204, the mobility of the patient during a second time period is monitored. The second time period may be a time period during which the patient has been prescribed one or more medications. For example, a patient may be prescribed one or more medications that are to be taken during the second time period. In some embodiments, the one or more medications prescribed to be taken by the patient during the second time period may be determined based on one or more health records associated with the patient, which are stored within health record database 134. In some embodiments, operation 1204 is performed by a subsystem that is the same or similar to location data collection subsystem 112, mobility detection subsystem 114, or a combination thereof.

In an operation 1206, a determination is made as to whether the patient took the one or more medications. The determination may be made based on a change in mobility of the patient during the first time period as compared to the second time period. In some embodiments, the determination is based on the change in the mobility of the patient between the first time period and the second time period being more than a threshold amount. The threshold amount may be a number of minutes, a number of hours, a percentage of a total amount of movement of the patient, etc. For example, the threshold amount may be a threshold value corresponding to a number, such as, without limitation, 10 minutes, 20 minutes, 1 hour (e.g., 60 minutes), 5%, etc. In some embodiments, one or more side effects of the one or more medications may be determined, and the determination of whether the patient took the one or more prescribed medications is based on the one or more side effects and the change in the mobility of the patient. In some embodiments, operation 1206 is performed by a subsystem that is the same or similar to mobility detection subsystem 114, warning detection subsystem 122, or a combination thereof.

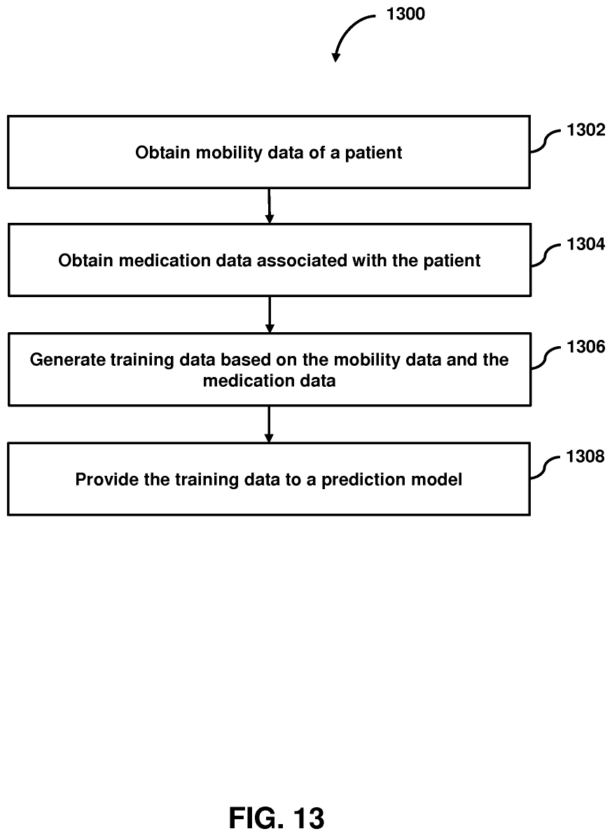

FIG. 13 shows another example method 1300 for facilitating training of a prediction model, in accordance with various embodiments. In an operation 1302, mobility data of a patient is obtained. In some embodiments, the mobility data may indicate movements of the patient during a first time period. The mobility data may be obtained from a wearable device, such as wearable device 104. which is configured to be worn by a patient of a care facility. The mobility data is configured to be stored, for example, in mobility database 138 with an indication of a corresponding patient (e.g., with a patient identifier or a wearable device identifier). Additionally or alternatively, the mobility data is obtained by determining an amount of movement of the patient during the first time period based on location data obtained from wearable device 104, where the location data includes a time series of locations of the patient during a first time period. The location data, for example, may be stored in location database 132 with an indication of the corresponding patient. In some embodiments, the mobility data includes an amount of movement of the patient during the first time period. For example, the mobility data may indicate that the patient experienced N minutes, hours, etc., of movement during a 24-hour time period. In some embodiments, operation 1302 is performed by a subsystem that is the same or similar to mobility detection subsystem 114.

In an operation 1304, medication data associated with the patient is obtained. The medication data may be obtained from health record database 134. In some embodiments, one or more health records associated with the patient are retrieved from health record database 134. The one or more health records may be analyzed to extract the medication data. The medication data, for example, may indicate a set of medications taken or prescribed to be taken by the patient during the first time period. The medication data may further indicate a sub-period within the first time period during which one or more medications (e.g., one or more medications of the set of medications), are taken or prescribed to be taken by the patient. In some embodiments, operation 1304 is performed by a subsystem that is the same or similar to medication data retrieval subsystem 116.

In an operation 1306, training data is generated based on the mobility data and the medication data. The training data may include the mobility data and the medication data. For example, the training data may include an amount of movement performed by the patient during the time period, as well as the set of medications taken or prescribed to be taken by the patient during the first time period. The training data can be stored in training data database 140, along with a timestamp indicating when the training data was generated. The timestamp may be used to determine whether a prediction model should employ the training data when being trained so as to ensure that the prediction model is trained using recently generated training data. In some embodiments, operation 1306 is performed by a subsystem that is the same or similar to model training subsystem 120.

In an operation 1308, the training data is provided to a prediction model. The training data may be provided to a prediction model, such as prediction model 810. In some embodiments, the prediction model (e.g., prediction model 810) is configured based on the training data. For example, prediction model 810 may be configured to estimate a dependency between one or more medications and a change in mobility. In some embodiments, the prediction model is a supervised machine learning model, a neural network, or another statistical model. After being configured, the prediction model may provide results indicating the estimated dependency, and the results may be stored within results database 144. In some embodiments, operation 1302 is performed by a subsystem that is the same or similar to model training subsystem 120.

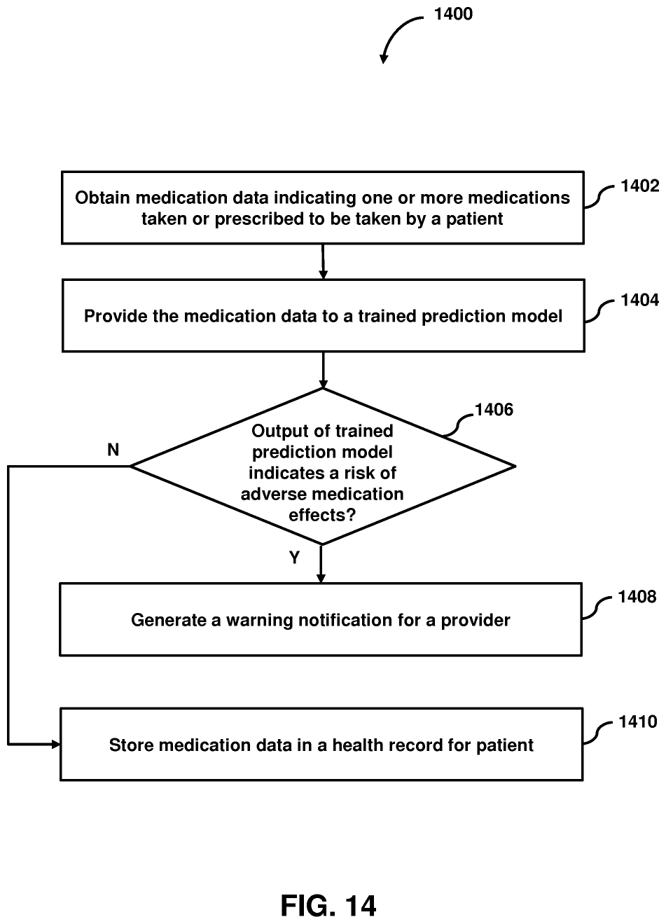

FIG. 14 shows an example method 1400 for generating a warning notification for potential adverse medication effects, in accordance with various embodiments. In an operation 1402, medication data indicating one or more medications taken or prescribed to be taken by a patient is obtained. The medication data may be obtained from health record database 134. In some embodiments, one or more health records associated with the patient are retrieved from health record database 134. The one or more health records may be analyzed to extract the medication data. The medication data, for example, may indicate a set of medications taken or prescribed to be taken by the patient during the first time period. The medication data may further indicate a sub-period within the first time period during which one or more medications (e.g., one or more medications of the set of medications), are taken or prescribed to be taken by the patient. In some embodiments, operation 1402 is performed by a subsystem that is the same or similar to medication data retrieval subsystem 116.

In an operation 1404, the medication data is provided to a trained prediction model. For instance, the medication data may be used as an input to a prediction model, such as prediction model 810. In some embodiments, the prediction model that is provided with the medication data has already been trained. For example, the prediction model may be trained to determine a likelihood that a medication or combination of medications cause one or more adverse medication effects (e.g., increased risk of fall events, decrease in mobility) to be experienced by a patient. In some embodiments, operation 1404 is performed by a subsystem that is the same or similar to warning detection subsystem 122.

In an operation 1406, a determination is made as to whether the output of the trained prediction model indicates a risk of adverse medication effects. For example, prediction model 810 is provided with the medication data as inputs 802, and returns outputs 804 indicating a likelihood that the one or more prescribed medications cause one or more adverse medication effects. In some embodiments, the medication data is retrieved from health record database 134 and is provided as inputs 802 to prediction model 810. In some embodiments, operation 1406 is performed by a subsystem that is the same or similar to model training subsystem 120, warning detection subsystem 122, or a combination thereof.

If the output of the trained prediction model indicates that there is a risk of adverse medication effects, method 1400 proceeds to operation 1408. In operation 1408, a warning notification for a provider is generated. The warning notification may indicate the risk that the one or more medications taken or prescribed to be taken by the patient causes the patient the one or more adverse medication effects. In some embodiments, the warning notification is generated in response to the output from the trained prediction model satisfying a threshold risk condition. For example, the output from the prediction model may be a risk value, and the threshold risk condition being satisfied may correspond to a determination made as to whether the risk value is equal to or greater than a threshold risk value. If so, the warning notification may be generated. In some embodiments, operation 1408 is performed by a subsystem that is the same or similar to warning detection subsystem 122.

If the output of the trained prediction model indicates that there is not a risk of adverse medication effects, method 1400 proceeds to operation 1410. In operation 1410, the medication data is stored in a health record for the patient. For instance, the one or more new medications taken or prescribed to be taken by the patient may be stored within a newly generated electronic health record, which is configured to be stored in health record database 134 in association with a patient identifier. In some embodiments, operation 1410 is performed by a subsystem that is the same or similar to medication data retrieval subsystem 116, warning detection subsystem 122, or a combination thereof.

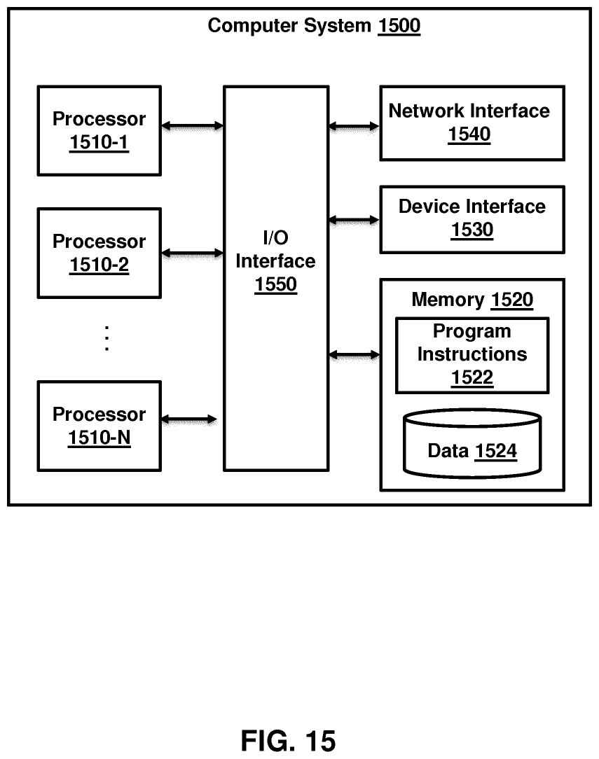

FIG. 15 shows an example computer system 1500 for implementing one or more of the aforementioned aspects, in accordance with various embodiments. Computer system 1500 may depict one or more components of wearable device(s) 104 and/or provider client device 106. In some embodiments, one or more components described by computer system 1500 may be excluded by wearable device(s) 104 and/or provider client device 106. Furthermore, one or more additional components may be included by wearable device(s) 104 and/or provider client device 106, and the foregoing is merely illustrative.

In some cases, multiple instances of computer system 1500 may communicate via a network to implement the present techniques in a distributed fashion. In some cases, instances may include a mobile computing device (like a smartphone with a camera) that captures images upon which the present patent application's techniques operate. In some cases, the instances may include server-side instances (e.g., in a micro-services architecture or monolithic architecture) that execute training and analysis with trained models. Various portions of systems and methods described herein, may include or be executed on one or more computer systems similar to computer system 1500. Further, processes and modules described herein may be executed by one or more processing systems similar to that of computer system 1500.

Computer system 1500 is configured to include one or more processors (e.g., processors 1510-1-1510-N) coupled to memory 1520, an input/output I/O device interface 1530, and a network interface 1540 via an input/output (I/O) interface 1550. As described herein, a processor can include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computer system 1500. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive computer program instructions and data from a memory (e.g., memory 1520). Computer system 1500 may be a uni-processor system including one processor (e.g., processor 1510a), or a multi-processor system including any number of suitable processors (e.g., 1510-1-1510-N). Multiple processors may be employed to provide for parallel or sequential execution of one or more portions of the techniques described herein. Processes, such as logic flows, described herein are capable of being performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computer system 1500 may include a plurality of computing devices (e.g., distributed computer systems) to implement various processing functions.

I/O device interface 1530 is configured to provide an interface for connection of one or more I/O devices, such as computer system 102, wearable device(s) 104, and/or provider client device 106, to computer system 1500. I/O devices may include devices that receive input (e.g., from a patient, provider) or output information (e.g., to a user, provider). I/O devices (e.g., provider client device) may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices may be connected to computer system 1500 through a wired or wireless connection. I/O devices may be connected to computer system 1500 from a remote location. I/O devices located on remote computer system, for example, may be connected to computer system 1500 via a network and network interface 1540.

Network interface 1540 may include a network adapter that provides for connection of computer system 1500 to a network. Network interface 1540 may facilitate data exchange between computer system 1500 and other devices connected to the network. Network interface 1540 may support wired or wireless communication. The network may include an electronic communication network, such as the Internet, a local area network (LAN), a wide area network (WAN), a cellular communications network, or the like.

System memory 1520 may be configured to store computer program instructions 1522 and/or data 1524. Computer program instructions 1522 may be executable by a processor (e.g., one or more of processors 1510-1-1510-N) to implement one or more embodiments of the present patent application's techniques. Computer program instructions 1522 may include modules of computer program instructions for implementing one or more techniques described herein with regard to various processing modules. Computer program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

Memory 1520 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. Memory 1520 may include a non-transitory computer readable storage medium that may have program instructions stored thereon that are executable by a computer processor (e.g., one or more of processors 1510-1-1510-N) to cause the subject matter and the functional operations described herein. A memory (e.g., memory 1520) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1550 may be configured to coordinate I/O traffic between processors 1510-1-1510-N, system memory 1520, network interface 1540, I/O devices (e.g., wearable device(s) 104, provider client device 106), and/or other peripheral devices. I/O interface 1550 may perform protocol, timing, or other data transformations to convert data signals from one component (e.g., memory 1520) into a format suitable for use by another component (e.g., processors 1510-1-1510-N). I/O interface 1550 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1500 or multiple computer systems 1500 configured to host different portions or instances of embodiments. Multiple computer systems 1500 may provide for parallel or sequential processing/execution of one or more portions of the techniques described herein.

Those skilled in the art will appreciate that computer system 1500 is merely illustrative and is not intended to limit the scope of the techniques described herein. Computer system 1500 may include any combination of devices or software that may perform or otherwise provide for the performance of the techniques described herein. For example, computer system 1500 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1500 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1500 may be transmitted to computer system 1500 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present patent application contemplates that, to the extent possible, one or more features of any embodiment can be combined with one or more features of any other embodiment.

The present techniques will be better understood with reference to the following enumerated embodiments:

A1. A method for training of a prediction model to estimate a fall risk due to medications, the method comprising: obtaining fall event data of a patient, wherein the fall event data comprises a set of fall events experienced by the patient during a first time period; obtaining medication data associated with the patient, wherein the medication data indicates a set of medications taken or prescribed to be taken by the patient during the first time period; generating training data for a prediction model based on the fall event data and the medication data; and providing the training data to the prediction model, the prediction model being configured, based on the training data, to estimate a dependency between one or more medications and an increase in a risk of experiencing a fall event.

A2. The method of embodiment A1, wherein obtaining the fall event data comprises: obtaining the fall event data from a patient client device worn by the patient during the first time period.

A3. The method of any of embodiments A1-A1, wherein obtaining the fall event data comprises: receiving data signals captured by a patient client device worn by the patient during the first time period, wherein the data signals indicate candidate fall events experienced by the patient during the first time period; determining, based on the data signals, one or more fall events from the candidate fall events; and generating the fall event data based on the one or more fall events, wherein the set of fall events comprises the one or more fall events.

A4. The method of embodiment A3, wherein determining the one or more fall events from the candidate fall events comprises: implementing a fall detection model configured to identify false fall events from the candidate fall events, and remove the false fall events from the candidate fall events to obtain the one or more fall events.

A5. The method of any of embodiments A1-A4, wherein obtaining the medication data comprises: receiving, for the patient, one or more health records from a health record database.

A6. The method of embodiment A5, further comprising: determining a medical condition or clinical status of the patient based on the one or more health records, wherein the training data generated further comprises information indicating the medical condition or clinical status of the patient.

A7. The method of any of embodiments A1-A6, wherein the first time period comprises a plurality of sub-periods, the method further comprises: determining a timestamp associated with each fall event of the set of fall events experienced by the patient during the first time period; determining a number of fall events experienced by the patient during each of the plurality of sub-periods based on the timestamp associated with each fall event of the set of fall events; and determining a sub-period of the plurality of sub-periods that each medication of the set of medications taken or prescribed to be taken by the patient, wherein the training data indicates (i) the number of fall events experienced by the patient during each of the plurality of sub-periods, and (ii) the sub-period that each medication of the set of medications is taken or prescribed to be taken by the patient.

A8. The method of any of embodiments A1-A7, wherein generating the training data comprises: generating the training data such that the training data comprises (i) a number of fall events experienced by the patient during the first time period and (ii) the set of medications or prescribed to be taken by the patient during the first time period.

A9. The method of any of embodiments A1-A7, further comprising: obtaining additional training data for the prediction model based on additional fall event data and additional medication data, wherein the additional fall event data comprises a plurality of sets of fall events experienced by a plurality of patients during a second time period, and the additional medication data comprises a plurality of sets of medications taken or prescribed to be taken by the plurality of patients during the second time period; and providing the additional training data to the prediction model, wherein the prediction model is configured, further based on the additional training data, to estimate the dependency.

B1. A method for facilitating training of a prediction model to estimate a change in mobility due to medications, the method comprising: obtaining mobility data of a patient, wherein the mobility data indicates movement of the patient during a first time period; obtaining medication data associated with the patient, wherein the medication data indicates a set of medications taken or prescribed to be taken by the patient during the first time period; generating training data for a prediction model based on the mobility data and the medication data; and providing the training data to the prediction model, the prediction model being configured, based on the training data, to estimate a dependency between one or more of the medications and a change in mobility.

B2. The method of embodiment B1, wherein obtaining the mobility data comprises: receiving location data indicating a location of a patient client device worn by the patient during the first time period; receiving sampling rate information related to a sampling rate with which the patient client device records the location of the patient client device; and generating the mobility data based on the location data and the sampling rate of the patient client device.

B3. The method of any of embodiments B1-B2, wherein the mobility data comprises an amount of movement of the patient during each of a plurality of sub-periods of the first time period, obtaining the medication data comprises: determining, based on a patient identifier of the patient, one or more health records associated with the patient; retrieving the one or more health records from a health record database; and generating the medication data by extracting information indicating the set of medications from the one or more health records, wherein the medication data indicates medications within the set of medications that are taken or prescribed to be taken by the patient during each of the plurality of sub-periods.

B4. The method of any of embodiments B1-B3, further comprising: determining, from the mobility data, an amount of movement of the patient during each of a plurality of sub-periods of the first time period, wherein the training data indicates (i) the amount of movement of the patient during each of the plurality of sub-periods and (ii) any medications within the set of medications taken or prescribed to be taken by the patient during each of the plurality of sub-periods.

B5. The method of any of embodiments B1-B4, wherein generating the training data comprises: generating the training data such that the training data comprises (i) the movement of the patient during the first time period and (ii) the set of medications taken or prescribed to be taken by the patient during the first time period.

B6. The method of any of embodiments B1-B5, further comprising: obtaining additional mobility data of the patient indicating movement of the patient during a second time period occurring prior to the first time period; obtaining additional medication data associated with the patient indicating a set of medications taken or prescribed to be taken by the patient during the second time period; and generating additional training data for the prediction model based on the additional mobility data and the additional medication data, wherein the additional training data is further provided to the prediction model such that the prediction is configured to estimate the dependency based on the training data and the additional training data.

C1. A method comprising: monitoring a mobility of a patient during a first time period when the patient has not been prescribed any medications; monitoring the mobility of the patient during a second time period when the patient has been prescribed one or more medications; and determining whether the patient took the one or more medications based upon a change in the mobility of the patient during the first time as compared to the mobility of the patient during the second time period.

C2. The method of embodiment C1, wherein determining whether the patient took the one or more medications is based on the change in the mobility between the first and second time periods being more than a threshold amount.

C3. The method of any of embodiments C1-C2, further comprising: obtaining medication data associated with the patient indicating the one or more medications taken or prescribed to be taken by the patient during second time period.

C4. The method of embodiment C3, wherein: the medication data indicates that a side effect of the one or more medications is an increase in mobility or decrease in mobility; and the determination of whether the mobility of the patient changed by the threshold amount is based on the side effect of the one or more medications being the increase in mobility or the decrease in mobility.

C5. The method of embodiment C4, wherein the side effect of the one or more medications is the increase in mobility, the patient is determined to have: taken the one or more medications if the mobility of the patient during second time period increased from mobility of the patient during the first time period; or not taken the one or more medications if the mobility of the patient during the second time period did not increase.

C6. The method of embodiment C4, wherein the side effect of the one or more medications is the decrease in mobility, the patient is determined to have: taken the one or more medications if the mobility of the patient during the second time period decreased from the mobility of the patient during the first time period; or not taken the one or more medications if the mobility of the patient during the second time period did not decrease.

C7. The method of any of embodiments C1-C6, further comprising: obtaining, from a wearable device, first mobility data indicating the mobility of the patient during the first time period; and obtaining, from the wearable, second mobility data indicating the mobility of the patient during the second time period, wherein the determination of whether the patient took the one or more medications based on the first mobility data and the second mobility data.

C8. The method of any of embodiments C1-C7, further comprising: generate a notification for a provider associated with the patient indicating whether the patient took the one or more medications during the second time period; and cause the notification to be provided to the provider.

D1. A method for generating a warning notification for potential adverse medication effects, the method comprising: obtaining medication data indicating a set of medications taken or prescribed to be taken by a first patient; providing the medication data to a trained prediction model; and generating a warning notification in response to an output from the trained prediction model indicating that a risk of the set of medications causing the patient adverse medication effects satisfies a threshold risk condition.

D2. The method of embodiment D1, wherein generating the warning notification comprises: determining that a first risk value associated with the risk is equal to or greater than a threshold risk value; causing the warning notification to be generated.

D3. The method of any of embodiments D1-D2, further comprising: providing the warning notification to a provider associated with the patient.

D4. The method of any of embodiments D1-D3, wherein obtaining the medication data comprises: receiving the medication data from a health record database subsequent to the first trained prediction model being trained.

D5. The method of any of embodiments D1-D4, wherein the risk satisfies the threshold risk condition, the method further comprises: determining whether the adverse medication effects comprise an increase in a risk of the patient experiencing a fall event, a risk of a decrease in a mobility of the patient, or the increase in the risk of the patient experiencing the fall event and the risk of the decrease in the mobility of the patient.

E1. A non-transitory computer readable medium comprising computer program instructions that, when executed by one or more processors, effectuate operations comprising a method of any of embodiments A1-A9, B1-B6, C1-C8, or D1-D5.

E2. A system, comprising: one or more wearable devices, each wearable device comprising one or more motion sensors, wherein the wearable device is to be worn by a patient; and a computer system comprising one or more processors operatively coupled to the wearable device and configured to execute computer program instructions such that, when executed, the one or more processors are configured to effectuate operations comprising a method of any of embodiments A1-A9, B1-B6, C1-C8, or D1-D5.

E3. A computer system, comprising: E2. one or more processors operatively coupled to the wearable device and configured to execute computer program instructions such that, when executed, the one or more processors are configured to effectuate operations comprising a method of any of embodiments A1-A9, B1-B6, C1-C8, or D1-D5.

1. A method for training of a prediction model to estimate a fall risk due to medications, the method being implemented by one or more processors executing one or more computer program instructions such that, when executed, the one or more processors effectuate the method, the method comprising:
obtaining fall event data of a patient, wherein the fall event data comprises a set of fall events experienced by the patient during a first time period;
obtaining medication data associated with the patient, wherein the medication data indicates a set of medications taken or prescribed to be taken by the patient during the first time period;
generating training data for a prediction model based on the fall event data and the medication data; and
providing the training data to the prediction model, the prediction model being configured, based on the training data, to estimate a dependency between one or morea combination of medications and an increase in a risk of experiencing a fall event.

2. The method of claim 1, wherein obtaining the fall event data comprises:
obtaining the fall event data from a patient client device worn by the patient during the first time period.

3. The method of claim 1, wherein obtaining the fall event data comprises:
receiving data signals detected by a wearable device worn by the patient during the first time period, wherein the data signals indicate candidate fall events experienced by the patient during the first time period;
determining, based on the data signals, one or more fall events from the candidate fall events; and
generating the fall event data based on the one or more fall events, wherein the set of fall events comprises the one or more fall events.

4. The method of claim 3, wherein determining the one or more fall events from the candidate fall events comprises:
implementing a fall detection model configured to identify false fall events from the candidate fall events, and remove the false fall events from the candidate fall events to obtain the one or more fall events.

5. The method of claim 1, wherein obtaining the medication data comprises:
receiving, for the patient, one or more health records from a health record database.

6. The method of claim 5, further comprising:
determining a medical condition or clinical status of the patient based on the one or more health records, wherein the training data generated further comprises information indicating the medical condition or clinical status of the patient.

7. The method of claim 1, wherein the first time period comprises a plurality of sub-periods, the method further comprises:
determining a timestamp associated with each fall event of the set of fall events experienced by the patient during the first time period;
determining a number of fall events experienced by the patient during each of the plurality of sub-periods based on the timestamp associated with each fall event of the set of fall events; and
determining a sub-period of the plurality of sub-periods that each medication of the set of medications taken or prescribed to be taken by the patient, wherein the training data indicates (i) the number of fall events experienced by the patient during each of the plurality of sub-periods, and (ii) the sub-period that each medication of the set of medications is taken or prescribed to be taken by the patient.

8. The method of claim 1, wherein generating the training data comprises:
generating the training data such that the training data comprises (i) a number of fall events experienced by the patient during the first time period and (ii) the set of medications or prescribed to be taken by the patient during the first time period.

9. The method of claim 1, further comprising:
obtaining additional training data for the prediction model based on additional fall event data and additional medication data, wherein the additional fall event data comprises a plurality of sets of fall events experienced by a plurality of patients during a second time period, and the additional medication data comprises a plurality of sets of medications to be taken by the plurality of patients during the second time period; and
providing the additional training data to the prediction model, wherein the prediction model is configured, further based on the additional training data, to estimate the dependency.

10. A non-transitory computer readable medium comprising computer program instructions that, when executed by one or more processors, effectuate operations comprising a method of claim 1.

11. A method for facilitating training of a prediction model to estimate a change in mobility due to medications, the method being implemented by one or more processors executing one or more computer program instructions such that, when executed, the one or more processors effectuate the method, the method comprising:
obtaining mobility data of a patient, wherein the mobility data indicates movement of the patient during a first time period;
obtaining medication data associated with the patient, wherein the medication data indicates a set of medications taken or prescribed to be taken by the patient during the first time period;
generating training data for a prediction model based on the mobility data and the medication data; and
providing the training data to the prediction model, the prediction model being configured, based on the training data, to estimate a dependency between one or more of the medications and a change in mobility.

12. The method of claim 11, wherein obtaining the mobility data comprises:
receiving location data indicating a location of a patient client device worn by the patient during the first time period;
receiving sampling rate information related to a sampling rate with which the patient client device records the location of the patient client device; and
generating the mobility data based on the location data and the sampling rate of the patient client device.

13. The method of claim 11, wherein the mobility data comprises an amount of movement of the patient during each of a plurality of sub-periods of the first time period, obtaining the medication data comprises:
determining, based on a patient identifier of the patient, one or more health records associated with the patient;
retrieving the one or more health records from a health record database; and
generating the medication data by extracting information indicating the set of medications from the one or more health records, wherein the medication data indicates medications within the set of medications that are to be taken by the patient during each of the plurality of sub-periods.

14. The method of claim 11, further comprising:
determining, from the mobility data, an amount of movement of the patient during each of a plurality of sub-periods of the first time period, wherein the training data indicates (i) the amount of movement of the patient during each of the plurality of sub-periods and (ii) any medications within the set of medications to be taken by the patient during each of the plurality of sub-periods.

15. The method of claim 11, wherein generating the training data comprises:
generating the training data such that the training data comprises (i) the movement of the patient during the first time period and (ii) the set of medications taken or prescribed to be taken by the patient during the first time period

16. The method of claim 11, further comprising:
obtaining additional mobility data of the patient indicating movement of the patient during a second time period occurring prior to the first time period;
obtaining additional medication data associated with the patient indicating a set of medications to be taken by the patient during the second time period; and
generating additional training data for the prediction model based on the additional mobility data and the additional medication data, wherein the additional training data is further provided to the prediction model such that the prediction is configured to estimate the dependency based on the training data and the additional training data.

17. A non-transitory computer readable medium comprising computer program instructions that, when executed by one or more processors, effectuate operations comprising a method of claim 11.

18. A system, comprising:
a wearable device comprising one or more motion sensors, wherein wearable device is to be worn by a patient; and
a computer system comprising one or more processors operatively coupled to the wearable device and configured to execute computer program instructions such that, when executed, the one or more processors are configured to:
monitor a mobility of a patient during a first time period when the patient has not been prescribed any medications;
monitor the mobility of the patient during a second time period when the patient has been prescribed one or more medications; and
determine whether the patient took the one or more medications based upon a change in the mobility of the patient during the first time period as compared to the mobility of the patient during the second time period.

19. The system of claim 18, wherein the determination of whether the patient took the one or more medications is based on the change in the mobility between the first time period and the second time period being more than a threshold amount.

20. The system of claim 18, wherein the one or more processors of the computer system are further configured by the computer program instructions, when executed, to:
obtain medication data associated with the patient indicating the one or more medications prescribed to be taken by the patient during second time period.

21. The system of claim 20, wherein:
the medication data indicates that a side effect of the one or more medications is an increase in mobility or decrease in mobility; and
the determination of whether the mobility of the patient changed by a threshold amount is based on the side effect of the one or more medications being the increase in mobility or the decrease in mobility.

22. The system of claim 21, wherein the side effect of the one or more medications is the increase in mobility, the patient is determined to have:
taken the one or more medications if the mobility of the patient during second time period increased from mobility of the patient during the first time period; or
not taken the one or more medications if the mobility of the patient during the second time period did not increase.

23. The system of claim 21, wherein the side effect of the one or more medications is the decrease in mobility, the patient is determined to have:
taken the one or more medications if the mobility of the patient during the second time period decreased from the mobility of the patient during the first time period; or
not taken the one or more medications if the mobility of the patient during the second time period did not decrease.

24. The system of claim 18, wherein the one or more processors of the computer system are further configured by the computer program instructions, when executed, to:
obtain, from the wearable device, first mobility data indicating the mobility of the patient during the first time period; and
obtain, from the wearable device, second mobility data indicating the mobility of the patient during the second time period, wherein the determination of whether the patient took the one or more medications based on the first mobility data and the second mobility data.

25. The system of claim 18, wherein the one or more processors of the computer system are further configured by the computer program instructions, when executed, to:
generate a notification for a provider associated with the patient indicating whether the patient took the one or more medications during the second time period; and
cause the notification to be provided to the provider.

26. A method for generating a warning notification for potential adverse medication effects, the method being implemented by one or more processors executing one or more computer program instructions such that, when executed, the one or more processors effectuate the method, the method comprising:
obtaining medication data indicating a set of medications taken or prescribed to be taken by a first patient;
providing the medication data to a trained prediction model, trained using the method of claim 1; and
generating a warning notification in response to an output from the trained prediction model indicating that a risk of the set of medications causing the patient adverse medication effects satisfies a threshold risk condition.

27. The method of claim 26, generating the warning notification comprises:
determining that a first risk value associated with the risk is equal to or greater than a threshold risk value;
causing the warning notification to be generated.

28. The method of claim 26, further comprising:
providing the warning notification to a provider associated with the patient.

29. The method of claim 26, wherein obtaining the medication data comprises:
receiving the medication data from a health record database subsequent to the first trained prediction model being trained.

30. The method of claim 26, wherein the risk satisfies the threshold risk condition, the method further comprises:
determining whether the adverse medication effects comprise an increase in a risk of the patient experiencing a fall event, a risk of a decrease in a mobility of the patient, or the increase in the risk of the patient experiencing the fall event and the risk of the decrease in the mobility of the patient.

31. A non-transitory computer readable medium comprising computer program instructions that, when executed by one or more processors, effectuate operations comprising a method of claim 26.
